(19) United States
(12) Patent Application Publication
(10) Pub. No.: US 2018/0275144 A1
(43) Pub. Date: Sep. 27, 2018

(54) DIAGNOSTIC TOOLS FOR ALZHEIMER'S DISEASE

(71) Applicant: PHARNEXT, ISSY LES MOULINEAUX (FR)

(72) Inventors: DANIEL COHEN, SAINT CLOUD (FR); ILYA CHUMAKOV, VAUX-LE-PENIL (FR); SERGUEI NABIROCHKIN, CHATENAY-MALABRY (FR); MICKAEL GUEDJ, PARIS (FR); RODOLPHE HAJJ, SAINT GERMAIN EN LAYE (FR)

(21) Appl. No.: 15/548,115

(22) PCT Filed: Feb. 2, 2016
(86) PCT No.: PCT/EP2016/052148

§ 371 Date: Aug. 2, 2017

(30) Foreign Application Priority Data
Feb. 3, 2015 (EP) 15305158.6

(51) Int. Cl. G01N 33/68; G01N 33/92

(52) U.S. Cl. CPC G01N 33/6896; G01N 33/92; G01N 2800/2814; G01N 2800/50; G01N 2570/00; G01N 2800/56; G01N 2800/2821

(57) ABSTRACT

The present invention relates to methods of detecting Alzheimer's disease using novel biomarkers or sets thereof. The novel biomarkers can be measured in biological body fluids or easily available extracts of biopsies.

### CROSS-REFERENCE TO RELATED APPLICATION

This application is the U.S. national stage application of International Patent Application No. PCT/EP2016/052148, filed Feb. 2, 2016.

### FIELD OF THE INVENTION

The present invention relates generally to the fields of biology and medicine. The present invention relates in particular to methods of detecting predisposition to or diagnosis and/or prognosis of Alzheimer's disease (AD) and related disorders. More specifically, the invention relates to the development, validation and application of new biomarkers, which can be used for detecting the presence, the risk, or for predicting the severity of AD and related disorders. The novel biomarkers can be measured in biological body fluids or easily available extracts of biopsies, which can be used to aid in the detection of neurodegenerative disorders, including AD. The present invention also relates to methods for identification of the stage of the disease in subjects having AD or a related disorder.

### BACKGROUND OF THE INVENTION

AD is at present the most common cause of dementia. It is clinically characterized by a global decline of cognitive function that progresses slowly and leaves end-stage patients bound to bed, incontinent and dependent on custodial care. Death occurs, on average, 9 years after diagnosis [1]. The incidence rate of AD increases dramatically with age. United Nation population projections estimate that the number of people older than 80 years will approach 370 million by the year 2050. Currently, it is estimated that 50% of people older than age 85 years are afflicted with AD. Therefore, more than 100 million people worldwide will suffer from dementia in 50 years. The vast number of people requiring constant care and other services will severely affect medical, monetary and human resources [2].

Currently, clinical diagnosis of AD is based on structured interviews (patient histories), and neuropsychological examinations coupled with imaging or 2 neurophysiological scans (CT, MM, PET and/or SPECT scans and EEG) to rule out other explanations of memory loss including temporary (depression or vitamin B12 deficiency) or permanent conditions (stroke) and is based on NINCDS-ADRDA Work group criteria [3] and the American Psychiatric Association Diagnostic and Statistical Manual of Mental Disorders [4].

Unfortunately, clinical diagnostic methods are not foolproof. Evidence based review of current literature shows clinical diagnostic accuracy of 65 to 90%. Higher accuracy rates are generally associated with specialized centers (memory disorder clinics) focused on memory disorders whereas lower rates are likely associated with primary care physicians. Additionally, accuracy of the clinical diagnosis is likely lower during early stages of the disease when symptoms are difficult to differentiate from normal age-associated cognitive decline. More recently, studies suggest that a condition termed Mild Cognitive Impairment (MCI) may represent in some cases a prodromal AD and, if diagnosed early, represents the best opportunity for pharmaceutical intervention. The clinical criteria used for diagnosis of MCI are those of Petersen et al. [5] and include: 1) memory complaints corroborated by an informant, 2) objective memory impairment for age and education, 3) normal general cognitive function, 4) intact activities of daily living, and 5) the subject does not meet criteria for dementia. This clinical criteria of MCI can be implemented with the identification of biomarkers such as those described in Albert et al. [6] and which are involved in neuronal injury (such as tau) and/or in Aβ deposition (such as Aβ42 in the Cerebro-Spinal Fluid). These biomarkers may be quantified through medical imaging and in the CSF. For instance, Amyvid is a FDA approved radioactive tracer that helps diagnosing AD by detecting amyloid plaques with the positron emission tomography imaging technology. This test, however, does neither allow predicting the development of AD nor measuring the response to the treatment and should only be used as an adjunct to other diagnostic evaluations to do this (FDA Press Release, Apr. 10, 2012).

Further complicating diagnosis and treatment of AD is the lack of a reliable biomarker that specifically identifies AD subjects and those at risk for a conversion from MCI to AD, particularly early in the prodromal stage of the disease (MCI). In view of the magnitude of the public health problem posed by AD, considerable research efforts have been undertaken to elucidate the etiology of AD as well as to identify biomarkers, characteristic proteins or metabolites objectively measured as an indicator of pathogenic processes, that can be used to diagnose and/or predict whether a person is likely to develop AD.

Most studies of biomarkers for AD have focused on measurement in the cerebrospinal fluid (CSF). Because of its intimate contact with the brain, pathogenic changes in the brain that result in alterations in proteins/peptides would likely be reflected in the CSF. Beside well known TAU, amyloid precursor protein derivatives, or neuronal thread protein, some CSF proteinaceous biomarkers described in the literature are alpha-(1)-antichymotrypsin, chromoganin A, β-2-microglobulin, transthyretin, cystatin C, transferritin or protaglandin-D-synthase; other studies measured proteinaceous biomarkers in biological fluids samples as blood (for instance US2010124756) but attempts to replicate the results of these studies failed [7]. Hence, it is not possible to derive from these studies a common set of biomarkers that could be considered a signature of the disease, certainly due in part to the heterogeneity and the complexity of the disease.

Some genetic biomarkers have been identified; they are localized within genetic loci which have been identified to be responsible for most cases of familial early-onset, autosomal-dominant AD. About sporadic AD, the most important identified genetic risk factor is the ApoE c4 allele: risk of developing AD is 12 times more important in homozygous people for ApoE c4 [8].

Metabolites as biomarkers for AD have also been searched. For instance, reduced levels of glutamate have been found in hippocampal cells of diseased patients using magnetic resonance spectroscopy, thus putting forward this molecule as a potential specific biomarker for AD [9]. Lipofuscin-like pigments, directly measurable from blood sample of patients, have been suggested as a possible specific marker of AD [10]. Aβ peptides blood tests have also been considered; nevertheless, until now, attempts to measure Aβ peptides in blood have produced contradictory and discouraging results mainly due to the biochemical nature of Aβ peptides. Indeed, Aβ can be found free in the plasma, bound to plasma proteins, to blood cells, either under soluble, or intracellular forms or in the form of deposits, and can also be generated from the outside of the CNS. Hence, the use of Aβ plasma levels as a biomarker needs further clinical and developmental researches [11-13].

WO2010/066000 discloses several blood or urine biomarkers identified from patients suffering from several mental diseases but not from AD. WO2011/012672 discloses some metabolites from disturbed pathways in AD. WO2012/168561 discloses notably some carboxylic acids containing 2 to 5 carbon atoms, phosphatidylcholine derivatives and unidentified serum metabolites for predicting the risk of subjects of progressing to AD.

Other fluid biomarkers in AD, blood-based protein biomarkers for diagnosis of AD and biochemical markers for early diagnosis of AD are also described [14-16].

The availability of reliable detectable biological markers would permit rapid diagnosis of AD and related diseases, patient monitoring, and efficient clinical testing of efficacy of new medications thanks to an easy monitoring of the individual response of patients to drug treatment and disease management.

### SUMMARY OF INVENTION

The present invention provides novel compositions and methods for diagnosing AD and related disorders. The invention stems from the identification of metabolites which represent effective biomarkers of the disease. The methods are effective, reliable, and easy to implement. They are particularly suited for diagnosing AD or related disorders from body fluids.

An object of the invention more particularly resides in a method for diagnosing AD or a related disorder, the method comprising determining the differential presence, in a sample from the subject, of one or more biomarker(s) selected from 5alpha-androstan-3alpha,17beta-diol monosulfate 1; 5alpha-androstan-3beta,17beta-diol monosulfate 2; 1-eicosapentaenoylglycerophosphocholine (20:5n3); 4-androsten-3beta,17beta-diol monosulfate 2; 1-eicosapentaenoylglycerophosphoethanolamine; 5alpha-androstan-3,17-diol monosulfate (alpha,beta or beta, alpha); C-glycosyltryptophan; 3-dehydrocarnitine; hydroxybutyrylcarnitine; taurocholenate sulfate; pregnen-diol disulfate; HWESASLLR; piperine; 3-[3-(sulfooxy)phenyl]propanoic acid; 3-hydroxyhippurate; methyl-beta-glucopyranoside; ergothioneine; salicylate; 3-carboxy-4-methyl-5-propyl-2-furanpropanoate (CMPF); pyruvate; 4-hydroxyhippurate; 4-methylcatechol sulfate; glycerate; N-acetyltyrosine; N2,N5-diacetylornithine; gamma-glutamylalanine; propionylglycine (C3); ornithine; N-acetylcarnosine; propionylcarnitine (C3); N-acetyltryptophan; gamma-glutamylmethionine; gamma-glutamylglutamate; alanine; isoleucylphenylalanine; gamma-glutamyllysine; lysine; uridine; histidine; methionine; carnitine; urate; leucine; isoleucine; nonadecanoate (19:0); 2-hydroxypalmitate; homovanillate sulfate; 3-methyl-2-oxobutyrate; succinate; citrate; myo-inositol; glycerol; fumarate; 5-hydroxyindoleacetate; adrenate (22:4n6); 17-methylstearate; acetylcarnitine (C2); adenine; dihomolinolenate (20:3n3 or 3n6); phenylalanylalanine; vaccenate (18:1n7); stearate (18:0); 1-methylguanosine; pentadecanoate (15:0); valylvaline; linolenate (18:3n3 or 3n6); taurine; N-acetylglycine; palmitoyl ethanolamide; glycolate (hydroxyacetate); palmitate (16:0); suberate (octanedioate); 13-methylmyristic acid; sphingosine 1-phosphate; a mix of 13-HODE and 9-HODE; phenylacetate; octadecanedioate (C18); asparagylleucine; methylpalmitate (15 or 2); N-palmitoyltaurine; docosapentaenoate (DPA; 22:5n3); glycylproline; linoleate (18:2n6); palmitoleate (16:1n7); margarate (17:0); docosadienoate (22:2n6); laurate (12:0); oleoylcarnitine (C18); palmitoylcarnitine (C16); 1-stearoylglycerol (18:0); isoleucylleucine; riboflavin (Vitamin B2); 6-oxopiperidine-2-carboxylic acid; valylglutamine; 3-methylglutaroylcarnitine (C6); oleate (18:1n9); myristate (14:0); caprylate (8:0); 10-heptadecenoate (17:1n7); dihydroferulic acid; 3-hydroxyoctanoate; threonylleucine; cysteine-glutathione disulfide; leucylglutamate; glutaroylcarnitine (C5); myristoleate (14:1n5); laurylcarnitine (C12); 5-dodecenoate (12:1n7); docosatrienoate (22:3n3); caprate (10:0); N-oleoyltaurine; 2-hydroxybutyrate (AHB); sarcosine (N-methylglycine); myristoylcarnitine; 10-nonadecenoate (19:1n9); 3-hydroxydecanoate; dihomolinoleate (20:2n6); eicosenoate (20:1n9 or 1n11); L-urobilin; 3-hydroxysebacate; hexadecanedioate (C16); leucylglycine; sphinganine; trimethylamine N-oxide; leucylalanine; tetradecanedioate (C14); iminodiacetate (IDA); taurolithocholate 3-sulfate; 3-hydroxybutyrate (BHBA); pyrophosphate (PPi); hypoxanthine; hippurate; tyrosine; tryptophan; undecanedioate; isovalerate (C5); 1-palmitoylglycerol (16:0); dodecanedioate (C12); sebacate (decanedioate); inosine, wherein said differential presence is indicative of the presence, risk, subtype, progression or severity of said disease.

In preferred embodiments, the method comprises the combined (simultaneous or sequential) detection of several biomarkers as listed above, preferably 2, 3, 4, 5, 6, 7, 8, 9, 10, more preferably 2 or 3, to provide the most effective patient analysis. In this regard, in a particular embodiment, the method of the invention comprises determining the differential presence, in a biological sample from the subject, of:

(i) one or more biomarker(s) selected from sarcosine (N-methylglycine), HWESASLLR, iminodiacetate (IDA), and 3-[3-(sulfooxy)phenyl]propanoic acid, and

(ii) one or more distinct biomarker(s) selected from sarcosine (N-methylglycine); HWESASLLR; iminodiacetate (IDA); 3-[3-(sulfooxy)phenyl]propanoic acid; leucylglycine; tetradecanedioate (C14); 3-hydroxybutyrate (BHBA); hexadecanedioate (C16); 3-dehydrocarnitine; caprate (10:0); threonylleucine; leucylglutamate; leucylalanine; N-oleoyltaurine; 2-hydroxybutyrate (AHB); a mix of 13-HODE and 9-HODE; sphinganine; hypoxanthine; glycolate (hydroxyacetate); taurocholenate sulfate; phenylacetate; myristate (14:0); margarate (17:0); valylglutamine; stearate (18:0); N-palmitoyltaurine; hydroxybutyrylcarnitine; glycerol; gamma-glutamylalanine; piperine; laurate (12:0); 10-nonadecenoate (19:1n9); dihomolinoleate (20:2n6); eicosenoate (20:1n9 or 1n11); lysine; 3-hydroxydecanoate; palmitate (16:0); 3-hydroxyhippurate; 5-dodecenoate (12:1n7); acetylcarnitine (C2); 5 alpha-androstan-3beta,17beta-diol monosulfate 2; methyl-beta-glucopyranoside; 1-eicosapentaenoylglycerophosphocholine (20:5n3); docosadienoate (22:2n6); gamma-glutamylmethionine; methylpalmitate (15 or 2); pentadecanoate (15:0); docosatrienoate (22:3n3); myristoylcarnitine; linoleate (18:2n6); 1-stearoylglycerol (18:0); myristoleate (14:1n5); oleate (18:1n9); 13-methylmyristic acid; nonadecanoate (19:0); 10-heptadecenoate (17:1n7); 5alpha-androstan-3,17-diol monosulfate (alpha,beta or beta, alpha); 17-methylstearate; vaccenate (18:1n7); palmitoleate (16:1n7); adrenate (22:4n6); linolenate (18:3n3 or 3n6); docosapentaenoate (DPA; 22:5n3); dihomolinolenate (20:3n3 or 3n6); adenine; 3-hydroxysebacate; N-acetyltyrosine; octadecanedioate (C18); isoleucylleucine; ergothioneine; N-acetylglycine; caprylate (8:0); citrate; N-acetyltryptophan; palmitoyl ethanolamide; histidine; asparagylleucine; 4-methylcatechol sulfate; suberate (octanedioate); methionine; cysteine-glutathione di sulfide; 6-oxopiperidine-2-carboxylic acid; glutaroylcarnitine (C5); taurolithocholate 3-sulfate; ornithine; palmitoylcarnitine (C16); 5alpha-androstan-3alpha,17beta-diol monosulfate 1; pyruvate; urate; 1-methylguanosine; C-glycosyltryptophan; 1-eicosapentaenoylglycerophosphoethanolamine; 3-hydroxyoctanoate; oleoylcarnitine (C18); sphingosine 1-phosphate; phenylalanylalanine; alanine; 3-methylglutaroylcarnitine (C6); N-acetylcarnosine; isoleucine; dihydroferulic acid; homovanillate sulfate; uridine; 4-hydroxyhippurate; leucine; glycylproline; trimethylamine N-oxide; laurylcarnitine (C12); propionylglycine (C3); propionylcarnitine (C3); fumarate; L-urobilin; glycerate; gamma-glutamyllysine; myo-inositol; pregnen-diol disulfate; 5-hydroxyindoleacetate; 2-hydroxypalmitate; 3-methyl-2-oxobutyrate; N2,N5-diacetylornithine; 4-androsten-3beta,17beta-diol monosulfate 2; taurine; valylvaline; gamma-glutamylglutamate; 3-carboxy-4-methyl-5-propyl-2-furanpropanoate (CMPF); carnitine; salicylate; succinate; isoleucylphenylalanine; riboflavin (Vitamin B2); pyrophosphate (PPi).

Most preferred biomarkers are selected from sarcosine (N-methylglycine); HWESASLLR; iminodiacetate (IDA); 3-[3-(sulfooxy)phenyl]propanoic acid; leucylglycine; tetradecanedioate (C14); 3-hydroxybutyrate (BHBA); hexadecanedioate (C16); 3-dehydrocarnitine; caprate (10:0); threonylleucine; leucylglutamate; leucylalanine; N-oleoyltaurine; 2-hydroxybutyrate (AHB); a mix of 13-HODE and 9-HODE; sphinganine; hypoxanthine; glycolate (hydroxyacetate); taurocholenate sulfate; phenylacetate; myristate (14:0); margarate (17:0); valylglutamine; stearate (18:0); N-palmitoyltaurine; hydroxybutyrylcarnitine; glycerol; gamma-glutamylalanine; piperine; laurate (12:0); 10-nonadecenoate (19:1n9); dihomolinoleate (20:2n6); eicosenoate (20:1n9 or 1n11); lysine; 3-hydroxydecanoate; palmitate (16:0); 3-hydroxyhippurate; 5-dodecenoate (12:1n7); acetylcarnitine (C2); 5 alpha-androstan-3beta,17beta-diol monosulfate 2; methyl-beta-glucopyranoside; 1-eicosapentaenoylglycerophosphocholine (20:5n3); docosadienoate (22:2n6); gamma-glutamylmethionine; methylpalmitate (15 or 2); pentadecanoate (15:0); docosatrienoate (22:3n3); myristoylcarnitine; linoleate (18:2n6); 1-stearoylglycerol (18:0); myristoleate (14:1n5); oleate (18:1n9); 13-methylmyristic acid; nonadecanoate (19:0); 10-heptadecenoate (17:1n7); 5alpha-androstan-3,17-diol monosulfate (alpha,beta or beta, alpha); 17-methylstearate; vaccenate (18:1n7); palmitoleate (16:1n7); adrenate (22:4n6); linolenate (18:3n3 or 3n6); docosapentaenoate (DPA; 22:5n3); dihomolinolenate (20:3n3 or 3n6).

Even more preferred biomarkers are selected from sarcosine (N-methylglycine); HWESASLLR; iminodiacetate (IDA); 3-[3-(sulfooxy)phenyl]propanoic acid; leucylglycine; tetradecanedioate (C14); 3-hydroxybutyrate (BHBA); hexadecanedioate (C16); 3-dehydrocarnitine; caprate (10:0); threonylleucine; leucylglutamate; leucylalanine; N-oleoyltaurine; 2-hydroxybutyrate (AHB); a mix of 13-HODE and 9-HODE; sphinganine; hypoxanthine; glycolate (hydroxyacetate); taurocholenate sulfate; phenylacetate; myristate (14:0); margarate (17:0); valylglutamine; stearate (18:0); N-palmitoyltaurine; hydroxybutyrylcarnitine; glycerol; gamma-glutamylalanine; piperine.

In another preferred embodiment, biomarkers are selected from sarcosine (N-methylglycine); HWESASLLR; iminodiacetate (IDA); 3-[3-(sulfooxy)phenyl]propanoic acid; leucylglycine; tetradecanedioate (C14); 3-hydroxybutyrate (BHBA); hexadecanedioate (C16); 3-dehydrocarnitine; caprate (10:0); threonylleucine; leucylglutamate.

The method may be implemented with any biological sample, typically a biological fluid, such as a sample of blood, plasma or serum. The sample may be treated prior to analysis.

A further object of the invention resides in a method for assessing the responsiveness of a subject to a treatment for AD or a related disorder, the method comprising determining the differential presence, in a biological fluid sample from the subject, of one or more biomarker(s) as defined above, after administration of said treatment, wherein said differential presence is indicative of a subject responsive to a treatment for AD or related disorder.

The invention also relates to a method for monitoring the effect of a treatment in a subject having AD or a related disorder, the method comprising determining the differential presence, in a biological fluid sample from the subject, of one or more biomarker(s) as defined above, after administration of said treatment or at different points of times during the course of the treatment, wherein a correction of such differential presence during treatment is indicative of an effective treatment. The method is particularly suited for determining the response of a subject having AD to a treatment by an acetylcholinesterase (AchE) inhibitor (for instance donepezil, tacrine, rivastigmine or galantamine) or an NMDA inhibitor (as memantine), or for monitoring efficacy of said treatment.

A further object of the invention is a method of treating a subject having or suspected to have AD or a related disorder, the method comprising (i) determining the presence, risk, subtype, progression or severity of said disease in a subject using a method as defined above and, (ii) administering to the subject in need thereof, a treatment against AD or said related disorder.

A further object of the invention is a kit comprising a capture/label agent specific for anyone of the biomarkers as defined above, for use in diagnosing AD or a related disorder in a subject.

The invention may be used in any mammalian, typically any human subject, at any stage of the disease.

### DETAILED DESCRIPTION OF THE INVENTION

The present invention discloses the identification of new biomarkers and diagnostic methods for Alzheimer's disease (AD) and related disorders. The invention describes novel use of biomarkers that can be detected in tissues and biological fluids for purposes of diagnosing AD and related disorders. More particularly, this invention relates to new metabolic biomarkers and combinations thereof useful to diagnose AD and related disorders.

### Definitions

Within the context of this invention, “AD related disorders” includes senile dementia of AD type (SDAT), prodromal AD, mild cognitive impairment (MCI), frontotemporal dementia (FTD), vascular dementia and age-associated memory impairment (AAMI).

It should nevertheless be contemplated that biomarkers of the invention, though particularly devoted to AD and related disorders, might find a use in diagnosing other neurological disorders that share some metabolic features with AD or related disorders, these are, for example, multiple sclerosis, Parkinson's disease or amyotrophic lateral sclerosis.

Within the context of the invention, diagnosing AD and related disorders means identifying or detecting or assessing a risk, presence, subtype, severity or progression of the pathologic condition. More particularly, diagnostic methods of the invention can be used to prognose the development of the disease, to detect the presence of the disease, to identify disease subtype, to monitor the progression of the disease, to qualify AD or related disorders, to assess the responsiveness of a subject to a treatment, to enhance patient stratification step in clinical trials, or to assess the efficacy of a treatment.

The term “biomarker” as used herein refers to any metabolite or molecule or analyte which can be used to diagnose a disorder in a subject, preferably a human subject, most preferably in a fluid sample from such a subject.

Metabolites are the downstream end products of genome, transcriptome and proteome variability of a biological system. Hence, the term “metabolite” encompasses any substance produced by the metabolism of an organism or by a metabolic process in an organism. For example, metabolites are small molecules as sugars, cholesterol, nucleosides, lipids, amino acids, or even peptides comprising 2 to 50 amino acids, preferably 2, 3, 4, 5, 6, 7, 8 or 9 amino acids.

The term “differentially present”, “differential presence” or “differential level” refers to an alteration in the presence, quantity and/or the frequency and/or form of a biomarker in a sample from a diseased subject as compared to a control. The differential presence therefore reflects the presence of a level (or frequency or form) which is different from a “normal” level. The control may be the quantity and/or the frequency and/or the form of the biomarker as determined in a similar sample from a healthy subject, or a reference value (e.g., median value, average value), and/or level(s) of the biomarker in a sample from the same subject before disease development and/or at an earlier stage of treatment/disease in the subject, and/or level(s) of the biomarker in a sample from another diseased subject or diseased subject population as control.

“Level” and “quantity” are interchangeable terms.

The terms “alteration” or “deviation” or “difference” in the quantity of a target biomarker may designate an increase or a decrease of the target biomarker quantity in a biological sample from the subject, in comparison with a control sample or reference value. Typically, the term “decrease” in relation to a biomarker level, designates a statistically significant reduction of the concentration or level of the biomarker in a biological sample from the subject. In an embodiment such a decrease is of at least 0.4%, 0.5%, 0.6%, 0.7%, 0.8%, 0.9% or 1% in comparison with a control sample or reference or mean value. In another embodiment such a decrease is of at least 1.5%, 2%, 2.5%, 3.0%, 3.5%, 4% or 4.5% in comparison with a control sample or reference or mean value. Decrease may be more substantial, such as a reduction by at least 5% or even more. In a preferred embodiment, decrease may be of about 10%, 15%, 20%, 30%, 40%, 50%, 60%, 70%, 80%, 90% or 100%. In a more preferred embodiment, decrease may be of about 2%, 5% or 15% or even more. Similarly, the term “increase” in relation to the biomarker level, designates a statistically significant augmentation of the concentration or level of the biomarker in a biological sample from the subject. In an embodiment, such an increase is of at least 0.4%, 0.5%, 0.6%, 0.7%, 0.8%, 0.9% or 1% in comparison with a control sample or reference or mean value. In another embodiment such an increase is of at least 1.5%, 2%, 2.5%, 3.0%, 3.5%, 4% or 4.5% in comparison with a control sample or reference or mean value. Increase may be more substantial, such as an increase by at least 5% or even more. In a preferred embodiment, increases may be of about 10%, 15%, 20%, 30%, 40%, 50%, 60%, 70%, 80%, 90% or 100% (or even more). In a more preferred embodiment, increase may be of about 2%, 5% or 15%. Alternatively, an alteration in the frequency of a biomarker can otherwise be observed. Said biomarker can be detected at a higher frequency or at a lower frequency in samples of patients compared to samples of control subjects. A biomarker can be differentially present in terms of quantity, frequency, and/or form, and is indicative of AD or related disorder in the subject. The order of magnitude of said increase or decrease may vary depending on the biomarker, patient, type or stage of disease. The order of variation in the level of biomarker (increase or decrease) as determined and disclosed in the present application is characteristic of the disease.

“Sensitivity”, “specifity” and “AUC” are statistical terms which are commonly used when talking about the predictive power of diagnostic kit. “Sensitivity” reflects the capacity of a test to give a positive result when the hypothesis is verified, and “specificity” the capacity to give a negative result when the hypothesis is rejected. Consequently, in the present invention, a high sensitivity means that the deviation of the biomarker is highly indicative of the disease onset, presence or progression; a high specificity means that the absence of a deviation of the biomarker is highly correlated to the absence of the onset, presence or progression of the disease. The Area under the ROC (Receiver Operating Characteristic) Curve (AUC) is the average sensitivity of the biomarker over the range of specificities. It is often used as a summary statistic representing the overall performance of the biomarker. A biomarker with no predictive value would have an AUC of 0.5 or less. As exemplified in the experimental section, biomarkers which have now been identified by the inventors are characteristic of AD and related disorders. More particularly, though being assayable in the CSF, biomarkers of the invention are metabolites which can also be assayed from body fluids that are more easily obtainable from the subject in comparison with the CSF.

Mining of data on AD and related disorders, new analysis of functional data and experimentations first allowed the inventors to identify pathways implied in the disease. These functional units were then combined and served as a starting point to construct larger functional networks of interacting pathways. Based on these networks, metabolites as candidate biomarkers could be identified and selected by the inventors. Such biomarkers were prioritized for different criteria, including:
their participation in a signaling pathway associated with onset and development of AD, and
their participation in the functional network cogently represented by AD-associated pathways.

This led to the identification of metabolites implicated in or interfering within several pathways thereby found to be altered in AD patients.

Further validation studies allowed the selection of valuable metabolite biomarkers that can be used alone, mixed together, or combined with other already known markers to diagnose AD or related disorders. The metabolites are characterized by their monoisotopic mass (Tables 1 and 2). The metabolites listed in Table 1 are those for which the identity has been further confirmed using the corresponding internal standard (when commercially available). These metabolite biomarkers were further tested to confirm their relevance to AD, as shown in the experimental section.

The metabolites are disclosed in Tables 1 and 2 below, with their name, monoisotopoic mass and, when available, the chemical formula (of the acid or base form) and illustrative CAS thereof.

TABLE 1

| | Monoisotopic | | |
|---|---|---|---|
| Metabolite name | mass (Da) | Molecular formula | Illustrative CAS number |
| 10-heptadecenoate (17:1n7) | 267.233 | C17H32O2 | 29743-97-3 |
| 10-nonadecenoate (19:1n9) | 295.2643 | C19H36O2 | 73033-09-7 |
| 13-HODE + 9-HODE | 295.2279 | C18H32O3 | N/A |
| 13-methylmyristic acid | 241.2173 | C15H30O2 | 2485-71-4 |
| 17-methylstearate | 297.2799 | C19H38O2 | 2724-59-6 |
| 1-methylguanosine | 298.1146 | C11H15N5O5 | 2140-65-0 |
| 1-palmitoylglycerol (16:0) | 330.27701 | C19H38O4 | 73299-28-2 |
| 1-stearoylglycerol (18:0) | 399.4 | C21H42O4 | 123-94-4; 31566-31-1 |
| 2-hydroxybutyrate (AHB) | 130.9 | C4H8O3 | 3347-90-8 |
| 2-hydroxypalmitate | 271.2279 | C16H32O3 | 764-67-0 |
| 3-[3-(sulfooxy)phenyl]propanoic acid | 245.0125 | C9H10O6S | 86321-32-6 |
| 3-carboxy-4-methyl-5-propyl-2- | 239.0925 | C12H16O5 | 86879-39-2 |
| furanpropanoate (CMPF) | | | |
| 3-hydroxybutyrate (BHBA) | 116.9 | C4H8O3 | 625-72-9 |
| 3-hydroxydecanoate | 187.134 | C10H20O3 | 5561-87-5 |
| 3-hydroxyhippurate | 194.0459 | C9H9NO4 | 1637-75-8 |
| 3-hydroxyoctanoate | 159.1027 | C8H16O3 | 88930-08-9 |
| 3-hydroxysebacate | 217.1081 | C10H18O5 | 73141-46-5 |
| 3-methyl-2-oxobutyrate | 115.0401 | C5H8O3 | 3715-29-5 |
| 3-methylglutaroylcarnitine (C6) | 290.1598 | C13H23NO6 | 102673-95-0 |
| 4-hydroxyhippurate | 194.0459 | C9H9NO4 | 2482-25-9 |
| 4-methylcatechol sulfate | 203.002 | C7H7O5S | N/A |
| 5-dodecenoate (12:1n7) | 197.1547 | C12H22O2 | 2430-94-6 |
| 5-hydroxyindoleacetate | 192.0655 | C10H9NO3 | 54-16-0 |
| 6-oxopiperidine-2-carboxylic acid | 142.051 | C6H9NO3 | 34622-39-4 |
| acetylcarnitine (C2) | 204.123 | C9H17NO4; | 5080-50-2 |
| | | C9H18NO4•Cl | |
| adenine | 136.0618 | C5H5N5 | 73-24-5 |
| adrenate (22:4n6) | 331.2643 | C22H36O2 | 2091-25-0 |
| alanine | 90.055 | C3H7NO2 | 56-41-7 |
| asparagylleucine | 246.1448 | C10H19N3O4 | N/A |
| caprate (10:0) | 171.1391 | C11H22O2 | 112-37-8 |
| caprylate (8:0) | 143.1078 | C8H16O2 | 124-07-2 |
| carnitine | 162.1125 | C7H15NO3; | 461-05-2 |
| | | C7H16NO3•Cl | |
| citrate | 273.1 | C6H8O7 | 77-92-9 |
| cysteine-glutathione disulfide | 427.0952 | C13H22N4O8S2 | 13081-14-6 |
| dihomolinoleate (20:2n6) | 307.2643 | C20H36O2 | 2091-39-6 |
| dihomolinolenate (20:3n3 or 3n6) | 305.2486 | C20H34O2 | 17046-59-2 |
| dihydroferulic acid | 195.0663 | C10H12O4 | 1135-23-5 |
| docosadienoate (22:2n6) | 335.2956 | C22H40O2 | 7370-49-2 |
| docosapentaenoate (DPA; 22:5n3) | 329.2486 | C22H34O2 | 2234-74-4 |
| docosatrienoate (22:3n3) | 230.15181 | C12H22O4 | 693-23-2 |
| dodecanedioate (C12) | 230.15181 | C12H22O4 | 693-23-2 |
| eicosenoate (20:1n9 or 1n11) | 309.2798 | C20H38O2 | 5561-99-9; 62322-84-3; |
| | | | 29204-02-2; 506-31-0 |
| ergothioneine | 230.0958 | C9H15N3O2S | 58511-63-0 |
| fumarate | 245 | C4H4O4 | 100-17-8 |
| gamma-glutamylalanine | 219.0976 | C8H14N2O5 | 5875-41-2 |
| gamma-glutamylglutamate | 277.103 | C10H16N2O7 | 1116-22-9 |
| gamma-glutamyllysine | 276.1554 | C11H21N3O5 | 17105-15-6 |
| gamma-glutamylmethionine | 279.1009 | C10H18N2O5S | 17663-87-5 |
| glutaroylcarnitine (C5) | 276.1442 | C12H21NO6 | 102636-82-8 |
| glycerate | 189 | C3H6O4 | 600-19-1 |
| glycerol | 205 | C3H8O3 | 56-81-5 |
| glycolate (hydroxyacetate) | 177 | C2H4O3 | 79-14-1 |
| glycylproline | 173.0921 | C7H12N2O3 | 704-15-4 |
| hexadecanedioate (C16) | 179.058244 | C9H9NO3 | 495-69-2 |
| hippurate | 179.058244 | C9H9NO3 | 495-69-2 |
| histidine | 154.0622 | C6H9N3O2•ClH•H2O | 5934-29-2 |
| homovanillate sulfate | 261.0074 | C9H8Na2O7S | 38339-06-9 |
| HWESASLLR | 549.7882 | N/A | N/A |
| hypoxanthine | 135.0312 | C5H4N4O | 68-94-0 |
| iminodiacetate (IDA) | 268.080771 | C10H12N4O5 | 58-63-9 |
| inosine | 268.080771 | C10H12N4O5 | 58-63-9 |
| isoleucine | 132.1019 | C6H13NO2 | 73-32-5 |
| isoleucylleucine | 245.186 | C12H24N2O3 | 26462-22-6 |
| isoleucylphenylalanine | 279.1703 | C15H22N2O3 | 22951-98-0 |
| isovalerate (C5) | 102.06808 | C5H10O2 | 503-74-2 |
| laurate (12:0) | 199.1704 | C12H24O2 | 143-07-7 |
| laurylcarnitine (C12) | 344.2795 | C19H37NO4 | 25518-54-1 |
| leucine | 132.1019 | C6H13NO2 | 61-90-5 |
| leucylalanine | 203.139 | C9H18N2O3 | 7298-84-2 |
| leucylglutamate | 261.1445 | C11H19N2O5 | N/A |
| leucylglycine | 189.1234 | C8H16N2O3 | 686-50-0 |
| linoleate (18:2n6) | 279.233 | C18H32O2 | 60-33-3 |
| linolenate (18:3n3 or 3n6) | 277.2173 | C18H30O2 | 463-40-1; 506-26-3 |
| L-urobilin | 595.349 | C33H46N4O6 | 34217-90-8 |
| lysine | 147.1128 | C6H14N2O2 | 56-87-1 |
| margarate (17:0) | 269.2486 | C17H34O2 | 506-12-7 |
| methionine | 150.0583 | C5H11NO2S | 63-68-3 |
| methyl-beta-glucopyranoside | 193.0718 | C7H14O6 | 709-50-2 |
| methylpalmitate (15 or 2) | 269.2486 | C17H34O2 | 1603-03-8; 27147-71-3 |
| myo-inositol | 217 | C6H12O6 | 87-89-8 |
| myristate (14:0) | 227.2017 | C14H28O2 | 544-63-8 |
| myristoleate (14:1n5) | 225.186 | C14H26O2 | 544-64-9 |
| myristoylcarnitine | 372.3108 | C21H42NO4Cl | 18822-89-4 |
| N2,N5-diacetylornithine | 215.1037 | C9H16N2O4 | 39825-23-5 |
| N-acetylcarnosine | 267.1099 | C11H16N4O4 | 56353-15-2 |
| N-acetylglycine | 116.0353 | C4H7NO3 | 543-24-8 |
| N-acetyltryptophan | 245.0932 | C13H14N2O3 | 1218-34-4 |
| N-acetyltyrosine | 222.0772 | C11H13NO4 | 537-55-3 |
| N-oleoyltaurine | 388.2527 | C20H39NO4S | 52514-04-2 |
| nonadecanoate (19:0) | 297.2799 | C19H38O2 | 646-30-0 |
| N-palmitoyltaurine | 362.2371 | C18H37NO4S | 83982-06-3 |
| octadecanedioate (C18) | 313.2384 | C18H34O4 | 871-70-5 |
| oleate (18:1n9) | 339.2 | C18H34O2 | 112-80-1 |
| oleoylcarnitine (C18) | 426.3578 | C25H47NO4 | 38677-66-6 |
| ornithine | 133.0972 | C5H12N2O2•ClH | 3184-13-2 |
| palmitate (16:0) | 255.233 | C16H32O2 | 57-10-3 |
| palmitoleate (16:1n7) | 253.2173 | C16H30O2 | 373-49-9 |
| palmitoyl ethanolamide | 298.2752 | C18H37NO2 | 544-31-0 |
| palmitoylcarnitine (C16) | 400.3421 | C23H46NO4•Cl | 6865-14-1 |
| pentadecanoate (15:0) | 241.2173 | C15H30O2 | 1002-84-2; 10002-84-2 |
| phenylacetate | 135.0452 | C8H8O2 | 103-82-2 |
| phenylalanylalanine | 237.1234 | C12H16N2O3 | 3918-87-4 |
| piperine | 286.1438 | C17H19NO3 | 94-62-2 |
| propionylcarnitine (C3) | 218.1387 | C10H19NO4 | 17298-37-2 |
| propionylglycine (C3) | 130.051 | C5H9NO3 | 21709-90-0 |
| pyrophosphate (PPi) | 451 | O7P2 | 1466-09-3 |
| pyruvate | 217 | C3H4O3 | 127-17-3 |
| riboflavin (Vitamin B2) | 377.1456 | C17H20N4O6 | 83-88-5 |
| salicylate | 202.12051 | C10H18O4 | 111-20-6 |
| sarcosine (N-Methylglycine) | 116 | C3H7NO2 | 107-97-1 |
| sebacate (decanedioate) | 202.12051 | C10H18O4 | 111-20-6 |
| sphinganine | 302.3054 | C18H39NO2 | 3102-56-5 |
| sphingosine 1-phosphate | 380.256 | C18H38NO5P | 26993-30-6 |
| stearate (18:0) | 283.2643 | C18H36O2 | 57-11-4 |
| suberate (octanedioate) | 173.0819 | C8H14O4 | 505-48-6 |
| succinate | 247 | C4H6O4 | 110-15-6 |
| taurine | 124.0074 | C2H7NO3S | 107-35-7 |
| taurolithocholate 3-sulfate | 280.6221 | C26H43NO8S2Na2 | 64936-83-0 |
| tetradecanedioate (C14) | 257.1758 | C14H26O4 | 821-38-5 |
| threonylleucine | 204.089878 | C11H12N2O2 | 73-22-3 |
| trimethylamine N-oxide | 181.073894 | C9H11NO3 | 60-18-4 |
| tryptophan | 216.13616 | C11H20O4 | 1852-04-6 |
| tyrosine | 181.073894 | C9H11NO3 | 60-18-4 |
| undecanedioate | 216.13616 | C11H20O4 | 1852-04-6 |
| urate | 167.0211 | C5H4N4O3 | 69-93-2 |
| uridine | 243.0623 | C9H12N2O6 | 58-96-8 |
| vaccenate (18:1n7) | 339.3 | C18H34O2 | 506-17-2 |
| valylglutamine | 244.1303 | C10H19N3O4 | 42854-54-6 |
| valylvaline | 217.1547 | C10H20N2O3 | 3918-94-3 |

N/A: not available

TABLE 2

| | | | Illustrative |
|---|---|---|---|
| | Monoisotopic | Molecular | CAS |
| Metabolite name | mass (Da) | formula | number |
| 5alpha-androstan-3alpha, | 371.1898 | C19H32O5S | N/A |
| 17beta-diol | | | |
| monosulfate 1 | | | |
| 5alpha-androstan-3beta, | 371.1898 | C19H32O5S | N/A |
| 17beta-diol | | | |
| monosulfate 2 | | | |
| 1-eicosapentaenoyl- | 542.3241 | C28H48NO7P | N/A |
| glycerophosphocholine | | | |
| (20:5n3) | | | |
| 4-androsten-3beta, | 369.1741 | C19H29O5S | N/A |
| 17beta-diol | | | |
| monosulfate 2 | | | |
| 1-eicosapentaenoyl- | 498.2626 | C25H47NO7P | N/A |
| glycerophosphoethanol- | | | |
| amine | | | |
| 5alpha-androstan-3,17- | 371.1898 | C19H32O5S | N/A |
| diol monosulfate | | | |
| (alpha,beta or beta,alpha) | | | |
| C-glycosyltryptophan | 367.15 | N/A | N/A |
| 3-dehydrocarnitine | 160.0968 | C7H13NO3 | 10457-99-5 |
| hydroxybutylylcarnitine | 248.1493 | C11H21NO5 | N/A |
| taurocholenate sulfate | 279.6142 | N/A | N/A |
| pregnen-diol disulfate | 238.0775 | N/A | N/A |

N/A: not available

The above metabolites represent valuable biomarkers which may be used, alone or in various combinations, for diagnosing AD or related disorders. The ability to detect and monitor levels of these biomarkers provides enhanced diagnostic capability by allowing clinicians to detect risk of developing disease in an early stage, to determine the level of the severity of the disease, to monitor the effects of the therapy by examination of these biomarkers in patient samples, or to sub-classify accurately patient in order, for example, to adapt the treatment or to predict the responsiveness of a patient to a treatment. In comparison to currently existing products, the invention provides several advantages and benefits. The herein described biomarkers provide more rapid, objective and accurate diagnosis of the disease or of its progression than existing diagnostic protocols. For example, neuropsychological tests (as Mini-Mental State Examination, MMSE) are only indicative of an impaired cognition and or dementia; their results can vary as a function of sociocultural factors and are generally taken as only indicative, when considered alone, of the presence or the absence of AD or a related disease. Furthermore, tools such as Amyvid, even if approved by the FDA, can be neither used as a predictive tool nor to appreciate the response to a treatment as stated by this administration.

The invention may be further used to predict the onset of AD and related disorders in advance of the appearance of any symptom conventionally used in the diagnostic of the disease. Thus the invention may be used in the testing and monitoring of individuals believed to be at risk of developing AD or a related disorder e.g. individuals with a family history of the disease, in order to enable early intervention to prevent onset or development of the symptoms. Such testing and monitoring may be used to identify or predict the development of AD and related disorders months or years in advance of the onset of the disease.

In other aspects, methods of the present invention further comprise the step of managing the individual treatment. For example, managing treatment comprises administering a matched drug or drug combination to slow, to halt or to reverse the progression of the disease. In another aspect of the invention, the method further comprises measuring the biomarker level after the treatment has begun, monitoring the progression of the disease, the response to the treatment or even the efficiency of the said selected treatment. In a particular embodiment monitoring the response to the treatment comprises determining the differential presence, in a biological fluid sample from the subject, of one or more of the above biomarkers, after administration of said treatment or at different point of times during the course of the treatment; a significant differential presence (whatever the order of variation) compared to the reference value being indicative of a response to the treatment.

As far as chronic diseases are concerned, in a particular embodiment, the monitoring of the response to the treatment comprises determining the differential presence, in a biological fluid from the subject, of one or more of the above biomarkers at different points of time during the course of the treatment.

In another particular embodiment, the monitoring of the disease progression comprises determining the differential presence, in a biological fluid from the subject, of one or more of the above biomarkers at different points of time during the course of the treatment.

In another particular embodiment, monitoring the efficiency of the treatment comprises determining the differential presence, in a biological fluid sample from the subject, of one or more of the above biomarkers, after administration of said treatment or at different point of times during the course of the treatment; a correction of such differential presence (i.e. an evolution toward a “normal state” level) during treatment being indicative of an effective treatment.

An object of the invention is a method for diagnosing AD or related disorders, which comprises detecting, measuring or determining the differential presence of at least one biomarker selected from sarcosine (N-methylglycine); HWESASLLR; iminodiacetate (IDA); 3-[3-(sulfooxy)phenyl]propanoic acid; leucylglycine; tetradecanedioate (C14); 3-hydroxybutyrate (BHBA); hexadecanedioate (C16); 3-dehydrocarnitine; caprate (10:0); threonylleucine; leucylglutamate; leucylalanine; N-oleoyltaurine; 2-hydroxybutyrate (AHB); a mix of 13-HODE and 9-HODE; sphinganine; hypoxanthine; glycolate (hydroxyacetate); taurocholenate sulfate; phenylacetate; myristate (14:0); margarate (17:0); valylglutamine; stearate (18:0); N-palmitoyltaurine; hydroxybutyrylcarnitine; glycerol; gamma-glutamylalanine; piperine; laurate (12:0); 10-nonadecenoate (19:1n9); dihomolinoleate (20:2n6); eicosenoate (20:1n9 or 1n11); lysine; 3-hydroxydecanoate; palmitate (16:0); 3-hydroxyhippurate; 5-dodecenoate (12:1n7); acetylcarnitine (C2); 5alpha-androstan-3beta,17beta-diol monosulfate 2; methyl-beta-glucopyranoside; 1-eicosapentaenoylglycerophosphocholine (20:5n3); docosadienoate (22:2n6); gamma-glutamylmethionine; methylpalmitate (15 or 2); pentadecanoate (15:0); docosatrienoate (22:3n3); myristoylcarnitine; linoleate (18:2n6); 1-stearoylglycerol (18:0); myristoleate (14:1n5); oleate (18:1n9); 13-methylmyristic acid; nonadecanoate (19:0); 10-heptadecenoate (17:1n7); 5alpha-androstan-3,17-diol monosulfate (alpha,beta or beta,alpha); 17-methylstearate; vaccenate (18:1n7); palmitoleate (16:1n7); adrenate (22:4n6); linolenate (18:3n3 or 3n6); docosapentaenoate (DPA; 22:5n3); dihomolinolenate (20:3n3 or 3n6); adenine; 3-hydroxysebacate; N-acetyltyrosine; octadecanedioate (C18); isoleucylleucine; ergothioneine; N-acetylglycine; caprylate (8:0); citrate; N-acetyltryptophan; palmitoyl ethanolamide; histidine; asparagylleucine; 4-methylcatechol sulfate; suberate (octanedioate); methionine; cysteine-glutathione disulfide; 6-oxopiperidine-2-carboxylic acid; glutaroylcarnitine (C5); taurolithocholate 3-sulfate; ornithine; palmitoylcarnitine (C16); 5 alpha-androstan-3 alpha,17beta-diol monosulfate 1; pyruvate; urate; 1-methylguanosine; C-glycosyltryptophan; 1-eicosapentaenoylglycerophosphoethanolamine; 3-hydroxyoctanoate; oleoylcarnitine (C18); sphingosine 1-phosphate; phenylalanylalanine; alanine; 3-methylglutaroylcarnitine (C6); N-acetylcarnosine; isoleucine; dihydroferulic acid; homovanillate sulfate; uridine; 4-hydroxyhippurate; leucine; glycylproline; trimethylamine N-oxide; laurylcarnitine (C12); propionylglycine (C3); propionylcarnitine (C3); fumarate; L-urobilin; glycerate; gamma-glutamyllysine; myo-inositol; pregnen-diol disulfate; 5-hydroxyindoleacetate; 2-hydroxypalmitate; 3-methyl-2-oxobutyrate; N2,N5-diacetylornithine; 4-androsten-3beta,17beta-diol monosulfate 2; taurine; valylvaline; gamma-glutamylglutamate; 3-carboxy-4-methyl-5-propyl-2-furanpropanoate (CMPF); carnitine; salicylate; succinate; isoleucylphenylalanine; riboflavin (Vitamin B2); pyrophosphate (PPi), in a mammal-derived sample, more preferably in a human-derived sample, such as differential presence being indicative of the disease.

More particularly, an object of this invention is a method for diagnosing AD or related disorder in a mammal, the method comprising determining the differential presence of at least one biomarker selected from sarcosine (N-methylglycine); HWESASLLR; iminodiacetate (IDA); 3-[3-(sulfooxy)phenyl]propanoic acid; leucylglycine; tetradecanedioate (C14); 3-hydroxybutyrate (BHBA); hexadecanedioate (C16); 3-dehydrocarnitine; caprate (10:0); threonylleucine; leucylglutamate; leucylalanine; N-oleoyltaurine; 2-hydroxybutyrate (AHB); a mix of 13-HODE and 9-HODE; sphinganine; hypoxanthine; glycolate (hydroxyacetate); taurocholenate sulfate; phenylacetate; myristate (14:0); margarate (17:0); valylglutamine; stearate (18:0); N-palmitoyltaurine; hydroxybutyrylcarnitine; glycerol; gamma-glutamylalanine; piperine; laurate (12:0); 10-nonadecenoate (19:1n9); dihomolinoleate (20:2n6); eicosenoate (20:1n9 or 1n11); lysine; 3-hydroxydecanoate; palmitate (16:0); 3-hydroxyhippurate; 5-dodecenoate (12:1n7); acetylcarnitine (C2); 5alpha-androstan-3beta,17beta-diol monosulfate 2; methyl-beta-glucopyranoside; 1-eicosapentaenoylglycerophosphocholine (20:5n3); docosadienoate (22:2n6); gamma-glutamylmethionine; methylpalmitate (15 or 2); pentadecanoate (15:0); docosatrienoate (22:3n3); myristoylcarnitine; linoleate (18:2n6); 1-stearoylglycerol (18:0); myristoleate (14:1n5); oleate (18:1n9); 13-methylmyristic acid; nonadecanoate (19:0); 10-heptadecenoate (17:1n7); 5alpha-androstan-3,17-diol monosulfate (alpha,beta or beta,alpha); 17-methylstearate; vaccenate (18:1n7); palmitoleate (16:1n7); adrenate (22:4n6); linolenate (18:3n3 or 3n6); docosapentaenoate (DPA; 22:5n3); dihomolinolenate (20:3n3 or 3n6), in a sample from the subject, such a differential presence being indicative of the disease.

Even more particularly, an object of this invention is a method for diagnosing AD or related disorder in a mammal, the method comprising determining the differential presence of at least one biomarker selected from sarcosine (N-methylglycine); HWESASLLR; iminodiacetate (IDA); 3-[3-(sulfooxy)phenyl]propanoic acid; leucylglycine; tetradecanedioate (C14); 3-hydroxybutyrate (BHBA); hexadecanedioate (C16); 3-dehydrocarnitine; caprate (10:0); threonylleucine; leucylglutamate; leucylalanine; N-oleoyltaurine; 2-hydroxybutyrate (AHB); a mix of 13-HODE and 9-HODE; sphinganine; hypoxanthine; glycolate (hydroxyacetate); taurocholenate sulfate; phenylacetate; myristate (14:0); margarate (17:0); valylglutamine; stearate (18:0); N-palmitoyltaurine; hydroxybutyrylcarnitine; glycerol; gamma-glutamylalanine; piperine, in a sample from the subject, such a differential presence being indicative of the disease.

Most particularly, an object of this invention is a method for diagnosing AD or related disorder in a mammal, the method comprising determining the differential presence of at least one biomarker selected from sarcosine (N-methylglycine); HWESASLLR; iminodiacetate (IDA); 3-[3-(sulfooxy)phenyl]propanoic acid; leucylglycine; tetradecanedioate (C14); 3-hydroxybutyrate (BHBA); hexadecanedioate (C16); 3-dehydrocarnitine; caprate (10:0); threonylleucine; leucylglutamate, in a sample from the subject, such a differential presence being indicative of the disease.

The sample may be, or may derive from, any metabolite-containing sample obtained from a subject such as a biological fluid, a gas, exhaled breath and/or aerosols, a biopsy, tissue extract, stool, etc. Preferably the sample is or derives from a biological fluid, such as interstitial, extracellular or intracellular fluid, more preferably from blood (or plasma and/or serum derived therefrom), urine, CSF, etc.

In this regard, according to a preferred embodiment, the method comprises determining the differential presence of at least one biomarker selected from sarcosine (N-methylglycine); HWESASLLR; iminodiacetate (IDA); 3-[3-(sulfooxy)phenyl]propanoic acid; leucylglycine; tetradecanedioate (C14); 3-hydroxybutyrate (BHBA); hexadecanedioate (C16); 3-dehydrocarnitine; caprate (10:0); threonylleucine; leucylglutamate; leucylalanine; N-oleoyltaurine; 2-hydroxybutyrate (AHB); a mix of 13-HODE and 9-HODE; sphinganine; hypoxanthine; glycolate (hydroxyacetate); taurocholenate sulfate; phenylacetate; myristate (14:0); margarate (17:0); valylglutamine; stearate (18:0); N-palmitoyltaurine; hydroxybutyrylcarnitine; glycerol; gamma-glutamylalanine; piperine; laurate (12:0); 10-nonadecenoate (19:1n9); dihomolinoleate (20:2n6); eicosenoate (20:1n9 or 1n11); lysine; 3-hydroxydecanoate; palmitate (16:0); 3-hydroxyhippurate; 5-dodecenoate (12:1n7); acetylcarnitine (C2); 5 alpha-androstan-3beta,17beta-diol monosulfate 2; methyl-beta-glucopyranoside; 1-eicosapentaenoylglycerophosphocholine (20:5n3); docosadienoate (22:2n6); gamma-glutamylmethionine; methylpalmitate (15 or 2); pentadecanoate (15:0); docosatrienoate (22:3n3); myristoylcarnitine; linoleate (18:2n6); 1-stearoylglycerol (18:0); myristoleate (14:1n5); oleate (18:1n9); 13-methylmyristic acid; nonadecanoate (19:0); 10-heptadecenoate (17:1n7); 5alpha-androstan-3,17-diol monosulfate (alpha,beta or beta,alpha); 17-methylstearate; vaccenate (18:1n7); palmitoleate (16:1n7); adrenate (22:4n6); linolenate (18:3n3 or 3n6); docosapentaenoate (DPA; 22:5n3); dihomolinolenate (20:3n3 or 3n6); adenine; 3-hydroxysebacate; N-acetyltyrosine; octadecanedioate (C18); isoleucylleucine; ergothioneine; N-acetylglycine; caprylate (8:0); citrate; N-acetyltryptophan; palmitoyl ethanolamide; histidine; asparagylleucine; 4-methylcatechol sulfate; suberate (octanedioate); methionine; cysteine-glutathione disulfide; 6-oxopiperidine-2-carboxylic acid; glutaroylcarnitine (C5); taurolithocholate 3-sulfate; ornithine; palmitoylcarnitine (C16); 5 alpha-androstan-3 alpha,17beta-diol monosulfate 1; pyruvate; urate; 1-methylguanosine; C-glycosyltryptophan; 1-eicosapentaenoylglycerophosphoethanolamine; 3-hydroxyoctanoate; oleoylcarnitine (C18); sphingosine 1-phosphate; phenylalanylalanine; alanine; 3-methylglutaroylcarnitine (C6); N-acetylcarnosine; isoleucine; dihydroferulic acid; homovanillate sulfate; uridine; 4-hydroxyhippurate; leucine; glycylproline; trimethylamine N-oxide; laurylcarnitine (C12); propionylglycine (C3); propionylcarnitine (C3); fumarate; L-urobilin; glycerate; gamma-glutamyllysine; myo-inositol; pregnen-diol disulfate; 5-hydroxyindoleacetate; 2-hydroxypalmitate; 3-methyl-2-oxobutyrate; N2,N5-diacetylornithine; 4-androsten-3beta,17beta-diol monosulfate 2; taurine; valylvaline; gamma-glutamylglutamate; 3-carboxy-4-methyl-5-propyl-2-furanpropanoate (CMPF); carnitine; salicylate; succinate; isoleucylphenylalanine; riboflavin (Vitamin B2); pyrophosphate (PPi), in a biological fluid from the subject, such a differential presence being indicative of the disease.

In a more preferred embodiment, the method comprises determining the differential presence of at least one biomarker selected from sarcosine (N-methylglycine); HWESASLLR; iminodiacetate (IDA); 3-[3-(sulfooxy)phenyl]propanoic acid; leucylglycine; tetradecanedioate (C14); 3-hydroxybutyrate (BHBA); hexadecanedioate (C16); 3-dehydrocarnitine; caprate (10:0); threonylleucine; leucylglutamate; leucylalanine; N-oleoyltaurine; 2-hydroxybutyrate (AHB); a mix of 13-HODE and 9-HODE; sphinganine; hypoxanthine; glycolate (hydroxyacetate); taurocholenate sulfate; phenylacetate; myristate (14:0); margarate (17:0); valylglutamine; stearate (18:0); N-palmitoyltaurine; hydroxybutyrylcarnitine; glycerol; gamma-glutamylalanine; piperine; laurate (12:0); 10-nonadecenoate (19:1n9); dihomolinoleate (20:2n6); eicosenoate (20:1n9 or 1n11); lysine; 3-hydroxydecanoate; palmitate (16:0); 3-hydroxyhippurate; 5-dodecenoate (12:1n7); acetylcarnitine (C2); 5alpha-androstan-3beta,17beta-diol monosulfate 2; methyl-beta-glucopyranoside; 1-eicosapentaenoylglycerophosphocholine (20:5n3); docosadienoate (22:2n6); gamma-glutamylmethionine; methylpalmitate (15 or 2); pentadecanoate (15:0); docosatrienoate (22:3n3); myristoylcarnitine; linoleate (18:2n6); 1-stearoylglycerol (18:0); myristoleate (14:1n5); oleate (18:1n9); 13-methylmyristic acid; nonadecanoate (19:0); 10-heptadecenoate (17:1n7); 5alpha-androstan-3,17-diol monosulfate (alpha,beta or beta,alpha); 17-methylstearate; vaccenate (18:1n7); palmitoleate (16:1n7); adrenate (22:4n6); linolenate (18:3n3 or 3n6); docosapentaenoate (DPA; 22:5n3); dihomolinolenate (20:3n3 or 3n6); adenine; 3-hydroxysebacate; N-acetyltyrosine; octadecanedioate (C18); isoleucylleucine; ergothioneine; N-acetylglycine; caprylate (8:0); citrate; N-acetyltryptophan; palmitoyl ethanolamide; histidine; asparagylleucine; 4-methyl catechol sulfate; suberate (octanedioate); methionine; cysteine-glutathione disulfide; 6-oxopiperidine-2-carboxylic acid; glutaroylcarnitine (C5); taurolithocholate 3-sulfate; ornithine; palmitoylcarnitine (C16); 5 alpha-androstan-3 alpha,17beta-diol monosulfate 1; pyruvate; urate; 1-methylguanosine; C-glycosyltryptophan; 1-eicosapentaenoylglycerophosphoethanolamine; 3-hydroxyoctanoate; oleoylcarnitine (C18); sphingosine 1-phosphate; phenylalanylalanine; alanine; 3-methylglutaroylcarnitine (C6); N-acetylcarnosine; isoleucine; dihydroferulic acid; homovanillate sulfate; uridine; 4-hydroxyhippurate; leucine; glycylproline; trimethylamine N-oxide; laurylcarnitine (C12); propionylglycine (C3); propionylcarnitine (C3); fumarate; L-urobilin; glycerate; gamma-glutamyllysine; myo-inositol; pregnen-diol disulfate; 5-hydroxyindoleacetate; 2-hydroxypalmitate; 3-methyl-2-oxobutyrate; N2,N5-diacetylornithine; 4-androsten-3beta,17beta-diol monosulfate 2; taurine; valylvaline; gamma-glutamylglutamate; 3-carboxy-4-methyl-5-propyl-2-furanpropanoate (CMPF); carnitine; salicylate; succinate; isoleucylphenylalanine; riboflavin (Vitamin B2); pyrophosphate (PPi), in blood, plasma and/or serum from the subject, such a differential presence being indicative of the disease.

In an even more preferred embodiment, an object of this invention is a method for diagnosing AD or related disorder in a mammal, the method comprising determining the differential presence of at least one biomarker selected from sarcosine (N-methylglycine); HWESASLLR; iminodiacetate (IDA); 3-[3-(sulfooxy)phenyl]propanoic acid; leucylglycine; tetradecanedioate (C14); 3-hydroxybutyrate (BHBA); hexadecanedioate (C16); 3-dehydrocarnitine; caprate (10:0); threonylleucine; leucylglutamate; leucylalanine; N-oleoyltaurine; 2-hydroxybutyrate (AHB); a mix of 13-HODE and 9-HODE; sphinganine; hypoxanthine; glycolate (hydroxyacetate); taurocholenate sulfate; phenylacetate; myristate (14:0); margarate (17:0); valylglutamine; stearate (18:0); N-palmitoyltaurine; hydroxybutyrylcarnitine; glycerol; gamma-glutamylalanine; piperine; laurate (12:0); 10-nonadecenoate (19:1n9); dihomolinoleate (20:2n6); eicosenoate (20:1n9 or 1n11); lysine; 3-hydroxydecanoate; palmitate (16:0); 3-hydroxyhippurate; 5-dodecenoate (12:1n7); acetylcarnitine (C2); 5 alpha-androstan-3beta,17beta-diol monosulfate 2; methyl-beta-glucopyranoside; 1-eicosapentaenoylglycerophosphocholine (20:5n3); docosadienoate (22:2n6); gamma-glutamylmethionine; methylpalmitate (15 or 2); pentadecanoate (15:0); docosatrienoate (22:3n3); myristoylcarnitine; linoleate (18:2n6); 1-stearoylglycerol (18:0); myristoleate (14:1n5); oleate (18:1n9); 13-methylmyristic acid; nonadecanoate (19:0); 10-heptadecenoate (17:1n7); 5alpha-androstan-3,17-diol monosulfate (alpha,beta or beta, alpha); 17-methylstearate; vaccenate (18:1n7); palmitoleate (16:1n7); adrenate (22:4n6); linolenate (18:3n3 or 3n6); docosapentaenoate (DPA; 22:5n3); dihomolinolenate (20:3n3 or 3n6), in blood, plasma and/or serum from the subject, such a differential presence being indicative of the disease.

In a most preferred embodiment, an object of this invention is a method for diagnosing AD or related disorder in a mammal, the method comprising determining the differential presence of at least one biomarker selected from sarcosine (N-methylglycine); HWESASLLR; iminodiacetate (IDA); 3-[3-(sulfooxy)phenyl]propanoic acid; leucylglycine; tetradecanedioate (C14); 3-hydroxybutyrate (BHBA); hexadecanedioate (C16); 3-dehydrocarnitine; caprate (10:0); threonylleucine; leucylglutamate; leucylalanine; N-oleoyltaurine; 2-hydroxybutyrate (AHB); a mix of 13-HODE and 9-HODE; sphinganine; hypoxanthine; glycolate (hydroxyacetate); taurocholenate sulfate; phenylacetate; myristate (14:0); margarate (17:0); valylglutamine; stearate (18:0); N-palmitoyltaurine; hydroxybutyrylcarnitine; glycerol; gamma-glutamylalanine; piperine, in blood, plasma and/or serum from the subject, such a differential presence being indicative of the disease.

In another preferred embodiment, an object of this invention is a method for diagnosing AD or related disorder in a mammal, the method comprising determining the differential presence of at least one biomarker selected from sarcosine (N-methylglycine); HWESASLLR; iminodiacetate (IDA); 3-[3-(sulfooxy)phenyl]propanoic acid; leucylglycine; tetradecanedioate (C14); 3-hydroxybutyrate (BHBA); hexadecanedioate (C16); 3-dehydrocarnitine; caprate (10:0); threonylleucine; leucylglutamate, in blood, plasma and/or serum from the subject, such a differential presence being indicative of the disease.

In another preferred embodiment, an object of this invention is a method for diagnosing AD or related disorder in a mammal, the method comprising determining the differential presence of at least one biomarker, selected from sarcosine (N-methylglycine); HWESASLLR; iminodiacetate (IDA); 3-[3-(sulfooxy)phenyl]propanoic acid; leucylglycine; tetradecanedioate (C14); 3-hydroxybutyrate (BHBA); hexadecanedioate (C16); 3-dehydrocarnitine; caprate (10:0); threonylleucine; leucylglutamate; leucylalanine; N-oleoyltaurine; 2-hydroxybutyrate (AHB); a mix of 13-HODE and 9-HODE; sphinganine; hypoxanthine; glycolate (hydroxyacetate); taurocholenate sulfate; phenylacetate; myristate (14:0); margarate (17:0); valylglutamine; stearate (18:0); N-palmitoyltaurine; hydroxybutyrylcarnitine; glycerol; gamma-glutamylalanine; piperine; laurate (12:0); 10-nonadecenoate (19:1n9); dihomolinoleate (20:2n6); eicosenoate (20:1n9 or 1n11); lysine; 3-hydroxydecanoate; palmitate (16:0); 3-hydroxyhippurate; 5-dodecenoate (12:1n7); acetylcarnitine (C2); 5 alpha-androstan-3beta,17beta-diol monosulfate 2; methyl-beta-glucopyranoside; 1-eicosapentaenoylglycerophosphocholine (20:5n3); docosadienoate (22:2n6); gamma-glutamylmethionine; methylpalmitate (15 or 2); pentadecanoate (15:0); docosatrienoate (22:3n3); myristoylcarnitine; linoleate (18:2n6); 1-stearoylglycerol (18:0); myristoleate (14:1n5); oleate (18:1n9); 13-methylmyristic acid; nonadecanoate (19:0); 10-heptadecenoate (17:1n7); 5alpha-androstan-3,17-diol monosulfate (alpha,beta or beta,alpha); 17-methylstearate; vaccenate (18:1n7); palmitoleate (16:1n7); adrenate (22:4n6); linolenate (18:3n3 or 3n6); docosapentaenoate (DPA; 22:5n3); dihomolinolenate (20:3n3 or 3n6); adenine; 3-hydroxysebacate; N-acetyltyrosine; octadecanedioate (C18); isoleucylleucine; ergothioneine; N-acetylglycine; caprylate (8:0); citrate; N-acetyltryptophan; palmitoyl ethanolamide; histidine; asparagylleucine; 4-methylcatechol sulfate; suberate (octanedioate); methionine; cysteine-glutathione disulfide; 6-oxopiperidine-2-carboxylic acid; glutaroylcarnitine (C5); taurolithocholate 3-sulfate; ornithine; palmitoylcarnitine (C16); alpha-androstan-3 alpha,17beta-diol monosulfate 1; pyruvate; urate; 1-methylguanosine; C-glycosyltryptophan; 1-eicosapentaenoylglycerophosphoethanolamine; 3-hydroxyoctanoate; oleoylcarnitine (C18); sphingosine 1-phosphate; phenylalanylalanine; alanine; 3-methylglutaroylcarnitine (C6); N-acetylcarnosine; isoleucine; dihydroferulic acid; homovanillate sulfate; uridine; 4-hydroxyhippurate; leucine; glycylproline; trimethylamine N-oxide; laurylcarnitine (C12); propionylglycine (C3); propionylcarnitine (C3); fumarate; L-urobilin; glycerate; gamma-glutamyllysine; myo-inositol; pregnen-diol disulfate; 5-hydroxyindoleacetate; 2-hydroxypalmitate; 3-methyl-2-oxobutyrate; N2,N5-diacetylornithine; 4-androsten-3beta,17beta-diol monosulfate 2; taurine; valylvaline; gamma-glutamylglutamate; 3-carboxy-4-methyl-5-propyl-2-furanpropanoate (CMPF); carnitine; salicylate; succinate; isoleucylphenylalanine; riboflavin (Vitamin B2); pyrophosphate (PPi), in the exhaled breath and/or aerosols from the subject, such a differential presence being indicative of the disease.

In an embodiment, diagnosing AD and related disorders, comprises the determination of the differential presence, in a biological fluid sample of the mammal, of one or more metabolite(s) selected from sarcosine (N-methylglycine); HWESASLLR; iminodiacetate (IDA); 3-[3-(sulfooxy)phenyl]propanoic acid; leucylglycine; tetradecanedioate (C14); 3-hydroxybutyrate (BHBA); hexadecanedioate (C16); 3-dehydrocarnitine; caprate (10:0); threonylleucine; leucylglutamate.

In a preferred embodiment, a method of the invention is an in vitro method for diagnosing AD or related disorders, the method comprising determining the differential presence of at least one biomarker selected from sarcosine (N-methylglycine); HWESASLLR; iminodiacetate (IDA); 3-[3-(sulfooxy)phenyl]propanoic acid; leucylglycine; tetradecanedioate (C14); 3-hydroxybutyrate (BHBA); hexadecanedioate (C16); 3-dehydrocarnitine; caprate (10:0); threonylleucine; leucylglutamate; leucylalanine; N-oleoyltaurine; 2-hydroxybutyrate (AHB); a mix of 13-HODE and 9-HODE; sphinganine; hypoxanthine; glycolate (hydroxyacetate); taurocholenate sulfate; phenylacetate; myristate (14:0); margarate (17:0); valylglutamine; stearate (18:0); N-palmitoyltaurine; hydroxybutyrylcarnitine; glycerol; gamma-glutamylalanine; piperine; laurate (12:0); 10-nonadecenoate (19:1n9); dihomolinoleate (20:2n6); eicosenoate (20:1n9 or 1n11); lysine; 3-hydroxydecanoate; palmitate (16:0); 3-hydroxyhippurate; 5-dodecenoate (12:1n7); acetylcarnitine (C2); 5alpha-androstan-3beta,17beta-diol monosulfate 2; methyl-beta-glucopyranoside; 1-eicosapentaenoylglycerophosphocholine (20:5n3); docosadienoate (22:2n6); gamma-glutamylmethionine; methylpalmitate (15 or 2); pentadecanoate (15:0); docosatrienoate (22:3n3); myristoylcarnitine; linoleate (18:2n6); 1-stearoylglycerol (18:0); myristoleate (14:1n5); oleate (18:1n9); 13-methylmyristic acid; nonadecanoate (19:0); 10-heptadecenoate (17:1n7); 5alpha-androstan-3,17-diol monosulfate (alpha,beta or beta,alpha); 17-methylstearate; vaccenate (18:1n7); palmitoleate (16:1n7); adrenate (22:4n6); linolenate (18:3n3 or 3n6); docosapentaenoate (DPA; 22:5n3); dihomolinolenate (20:3n3 or 3n6); adenine; 3-hydroxysebacate; N-acetyltyrosine; octadecanedioate (C18); isoleucylleucine; ergothioneine; N-acetylglycine; caprylate (8:0); citrate; N-acetyltryptophan; palmitoyl ethanolamide; histidine; asparagylleucine; 4-methylcatechol sulfate; suberate (octanedioate); methionine; cysteine-glutathione di sulfide; 6-oxopiperidine-2-carboxylic acid; glutaroylcarnitine (C5); taurolithocholate 3-sulfate; ornithine; palmitoylcarnitine (C16); 5 alpha-androstan-3 alpha,17beta-diol monosulfate 1; pyruvate; urate; 1-methylguanosine; C-glycosyltryptophan; 1-eicosapentaenoylglycerophosphoethanolamine; 3-hydroxyoctanoate; oleoylcarnitine (C18); sphingosine 1-phosphate; phenylalanylalanine; alanine; 3-methylglutaroylcarnitine (C6); N-acetylcarnosine; isoleucine; dihydroferulic acid; homovanillate sulfate; uridine; 4-hydroxyhippurate; leucine; glycylproline; trimethylamine N-oxide; laurylcarnitine (C12); propionylglycine (C3); propionylcarnitine (C3); fumarate; L-urobilin; glycerate; gamma-glutamyllysine; myo-inositol; pregnen-diol disulfate; 5-hydroxyindoleacetate; 2-hydroxypalmitate; 3-methyl-2-oxobutyrate; N2,N5-diacetylornithine; 4-androsten-3beta,17beta-diol monosulfate 2; taurine; valylvaline; gamma-glutamyl glutamate; 3-carboxy-4-methyl-5-propyl-2-furanpropanoate (CMPF); carnitine; salicylate; succinate; isoleucylphenylalanine; riboflavin (Vitamin B2); pyrophosphate (PPi), in a biological fluid sample from the subject, wherein said differential presence is indicative of the presence, risk, subtype, progression or severity of said disease.

In a more preferred embodiment, diagnosing AD or related disorders comprises measuring, in a biological fluid sample of the mammal, an increase of at least one biomarker selected from sarcosine (N-methylglycine); iminodiacetate (IDA); leucylglycine; tetradecanedioate (C14); 3-hydroxybutyrate (BHBA); hexadecanedioate (C16); 3-dehydrocarnitine; caprate (10:0); threonylleucine; leucylglutamate; leucylalanine; N-oleoyltaurine; 2-hydroxybutyrate (AHB); a mix of 13-HODE and 9-HODE; sphinganine; hypoxanthine; glycolate (hydroxyacetate); taurocholenate sulfate; phenylacetate; myristate (14:0); margarate (17:0); valylglutamine; stearate (18:0); N-palmitoyltaurine; hydroxybutyrylcarnitine; glycerol; laurate (12:0); 10-nonadecenoate (19:1n9); dihomolinoleate (20:2n6); eicosenoate (20:1n9 or 1n11); 3-hydroxydecanoate; palmitate (16:0); 5-dodecenoate (12:1n7); acetylcarnitine (C2); docosadienoate (22:2n6); methylpalmitate (15 or 2); pentadecanoate (15:0); docosatrienoate (22:3n3); myristoylcarnitine; linoleate (18:2n6); 1-stearoylglycerol (18:0); myristoleate (14:1n5); oleate (18:1n9); 13-methylmyristic acid; nonadecanoate (19:0); 10-heptadecenoate (17:1n7); 17-methylstearate; vaccenate (18:1n7); palmitoleate (16:1n7); adrenate (22:4n6); linolenate (18:3n3 or 3n6); docosapentaenoate (DPA; 22:5n3); dihomolinolenate (20:3n3 or 3n6); adenine; 3-hydroxysebacate; octadecanedioate (C18); isoleucylleucine; N-acetylglycine; caprylate (8:0); citrate; palmitoyl ethanolamide; asparagylleucine; suberate (octanedioate); cysteine-glutathione disulfide; 6-oxopiperidine-2-carboxylic acid; glutaroylcarnitine (C5); taurolithocholate 3-sulfate; palmitoylcarnitine (C16); 1-methylguanosine; C-glycosyltryptophan; 3-hydroxyoctanoate; oleoylcarnitine (C18); sphingosine 1-phosphate; phenylalanylalanine; 3-methylglutaroylcarnitine (C6); dihydroferulic acid; homovanillate sulfate; glycylproline; trimethylamine N-oxide; laurylcarnitine (C12); fumarate; L-urobilin; myo-inositol; pregnen-diol di sulfate; 5-hydroxyindoleacetate; 2-hydroxypalmitate; 3-methyl-2-oxobutyrate; taurine; valylvaline; succinate; riboflavin (Vitamin B2); pyrophosphate (PPi), and/or a decrease of at least one biomarker selected from HWESASLLR; 3-[3-(sulfooxy)phenyl]propanoic acid; gamma-glutamylalanine; piperine; lysine; 3-hydroxyhippurate; 5 alpha-androstan-3beta,17beta-diol monosulfate 2; methyl-beta-glucopyranoside; 1-eicosapentaenoylglycerophosphocholine (20:5n3); gamma-glutamylmethionine; 5alpha-androstan-3,17-diol monosulfate (alpha,beta or beta,alpha); N-acetyltyrosine; ergothioneine; N-acetyltryptophan; histidine; 4-methylcatechol sulfate; methionine; ornithine; 5alpha-androstan-3alpha,17beta-diol monosulfate 1; pyruvate; urate; 1-eicosapentaenoylglycerophosphoethanolamine; alanine; N-acetylcarnosine; isoleucine; uridine; 4-hydroxyhippurate; leucine; propionylglycine (C3); propionylcarnitine (C3); glycerate; gamma-glutamyllysine; N2,N5-diacetylornithine; 4-androsten-3beta,17beta-diol monosulfate 2; gamma-glutamyl glutamate; 3-carboxy-4-methyl-5-propyl-2-furanpropanoate (CMPF); carnitine; salicylate; isoleucylphenylalanine.

In a very preferred embodiment, diagnosing AD or related disorders comprises measuring, in a biological fluid sample of the mammal, an increase of at least one biomarker selected from iminodiacetate (IDA); sarcosine (N-methylglycine); leucylglycine; tetradecanedioate (C14); 3-hydroxybutyrate (BHBA); hexadecanedioate (C16); 3-dehydrocarnitine; caprate (10:0); threonylleucine; leucylglutamate, and/or a decrease of at least one biomarker selected from HWESASLLR; 3-[3-(sulfooxy)phenyl]propanoic acid.

In a particular embodiment, the invention relates to an in vitro method for diagnosing a neurological disease selected from Alzheimer's disease (AD), senile dementia of AD type, prodromal AD, mild cognitive impairment, age associated memory impairment, vascular dementia or frontotemporal dementia, said method comprising the following steps:
collecting blood, serum or plasma sample from a subject suffering from, or suspected to suffer from, or at risk of suffering from said disease,
treating samples for their further analysis by LC/MS and/or GC/MS,
measuring by LC/MS and/or GC/MS an increase, as compared to a control value, of at least one biomarker selected from iminodiacetate (IDA); sarcosine (N-methylglycine); leucylglycine; tetradecanedioate (C14); 3-hydroxybutyrate (BHBA); hexadecanedioate (C16); 3-dehydrocarnitine; caprate (10:0); threonylleucine; leucylglutamate; leucylalanine; N-oleoyltaurine; 2-hydroxybutyrate (AHB); a mix of 13-HODE and 9-HODE; sphinganine; hypoxanthine; glycolate (hydroxyacetate); taurocholenate sulfate; phenylacetate; myristate (14:0); margarate (17:0); valylglutamine; stearate (18:0); N-palmitoyltaurine; hydroxybutyrylcarnitine; glycerol; laurate (12:0); 10-nonadecenoate (19:1n9); dihomolinoleate (20:2n6); eicosenoate (20:1n9 or 1n11); 3-hydroxydecanoate; palmitate (16:0); 5-dodecenoate (12:1n7); acetylcarnitine (C2); docosadienoate (22:2n6); methylpalmitate (15 or 2); pentadecanoate (15:0); docosatrienoate (22:3n3); myristoylcarnitine; linoleate (18:2n6); 1-stearoylglycerol (18:0); myristoleate (14:1n5); oleate (18:1n9); 13-methylmyristic acid; nonadecanoate (19:0); 10-heptadecenoate (17:1n7); 17-methylstearate; vaccenate (18:1n7); palmitoleate (16:1n7); adrenate (22:4n6); linolenate (18:3n3 or 3n6); docosapentaenoate (DPA; 22:5n3); dihomolinolenate (20:3n3 or 3n6); adenine; 3-hydroxysebacate; octadecanedioate (C18); isoleucylleucine; N-acetylglycine; caprylate (8:0); citrate; palmitoyl ethanolamide; asparagylleucine; suberate (octanedioate); cysteine-glutathione disulfide; 6-oxopiperidine-2-carboxylic acid; glutaroylcarnitine (C5); taurolithocholate 3-sulfate; palmitoylcarnitine (C16); 1-methylguanosine; C-glycosyltryptophan; 3-hydroxyoctanoate; oleoylcarnitine (C18); sphingosine 1-phosphate; phenylalanylalanine; 3-methylglutaroylcarnitine (C6); dihydroferulic acid; homovanillate sulfate; glycylproline; trimethylamine N-oxide; laurylcarnitine (C12); fumarate; L-urobilin; myo-inositol; pregnen-diol di sulfate; 5-hydroxyindoleacetate; 2-hydroxypalmitate; 3-methyl-2-oxobutyrate; taurine; valylvaline; succinate; riboflavin (Vitamin B2); pyrophosphate (PPi), and/or a decrease, as compared to a control value, of at least one biomarker selected from HWESASLLR; 3-[3-(sulfooxy)phenyl]propanoic acid; gamma-glutamylalanine; piperine; lysine; 3-hydroxyhippurate; 5 alpha-androstan-3beta,17beta-diol monosulfate 2; methyl-beta-glucopyranoside; 1-eicosapentaenoylglycerophosphocholine (20:5n3); gamma-glutamylmethionine; 5alpha-androstan-3,17-diol monosulfate (alpha,beta or beta,alpha); N-acetyltyrosine; ergothioneine; N-acetyltryptophan; histidine; 4-methylcatechol sulfate; methionine; ornithine; 5alpha-androstan-3alpha,17beta-diol monosulfate 1; pyruvate; urate; 1-eicosapentaenoylglycerophosphoethanolamine; alanine; N-acetylcarnosine; isoleucine; uridine; 4-hydroxyhippurate; leucine; propionylglycine (C3); propionylcarnitine (C3); glycerate; gamma-glutamyllysine; N2,N5-diacetylornithine; 4-androsten-3beta,17beta-diol monosulfate 2; gamma-glutamyl glutamate; 3-carboxy-4-methyl-5-propyl-2-furanpropanoate (CMPF); carnitine; salicylate; isoleucylphenylalanine.
deducing from the preceding step the presence, risk, subtype, progression or severity of said disease.

In an even more preferred embodiment, methods for diagnosing AD or related disorders of the present invention comprise determining the differential presence of a combination of several biomarkers of the present invention, named set of biomarkers. A set contains preferably 2, 3, 4 or 5 (or even more) biomarkers from the above listed biomarkers, which may be determined simultaneously or sequentially in the sample.

In another embodiment, this set of biomarkers is constituted of at least two metabolites selected from the group comprising sarcosine (N-methylglycine); HWESASLLR; iminodiacetate (IDA); 3-[3-(sulfooxy)phenyl]propanoic acid; leucylglycine; tetradecanedioate (C14); 3-hydroxybutyrate (BHBA); hexadecanedioate (C16); 3-dehydrocarnitine; caprate (10:0); threonylleucine; leucylglutamate; leucylalanine; N-oleoyltaurine; 2-hydroxybutyrate (AHB); a mix of 13-HODE and 9-HODE; sphinganine; hypoxanthine; glycolate (hydroxyacetate); taurocholenate sulfate; phenylacetate; myristate (14:0); margarate (17:0); valylglutamine; stearate (18:0); N-palmitoyltaurine; hydroxybutyrylcarnitine; glycerol; gamma-glutamylalanine; piperine; laurate (12:0); 10-nonadecenoate (19:1n9); dihomolinoleate (20:2n6); eicosenoate (20:1n9 or 1n11); lysine; 3-hydroxydecanoate; palmitate (16:0); 3-hydroxyhippurate; 5-dodecenoate (12:1n7); acetylcarnitine (C2); 5 alpha-androstan-3beta,17beta-diol monosulfate 2; methyl-beta-glucopyranoside; 1-eicosapentaenoylglycerophosphocholine (20:5n3); docosadienoate (22:2n6); gamma-glutamylmethionine; methylpalmitate (15 or 2); pentadecanoate (15:0); docosatrienoate (22:3n3); myristoylcarnitine; linoleate (18:2n6); 1-stearoylglycerol (18:0); myristoleate (14:1n5); oleate (18:1n9); 13-methylmyristic acid; nonadecanoate (19:0); 10-heptadecenoate (17:1n7); 5alpha-androstan-3,17-diol monosulfate (alpha,beta or beta, alpha); 17-methylstearate; vaccenate (18:1n7); palmitoleate (16:1n7); adrenate (22:4n6); linolenate (18:3n3 or 3n6); docosapentaenoate (DPA; 22:5n3); dihomolinolenate (20:3n3 or 3n6); adenine; 3-hydroxysebacate; N-acetyltyrosine; octadecanedioate (C18); isoleucylleucine; ergothioneine; N-acetylglycine; caprylate (8:0); citrate; N-acetyltryptophan; palmitoyl ethanolamide; histidine; asparagylleucine; 4-methylcatechol sulfate; suberate (octanedioate); methionine; cysteine-glutathione di sulfide; 6-oxopiperidine-2-carboxylic acid; glutaroylcarnitine (C5); taurolithocholate 3-sulfate; ornithine; palmitoylcarnitine (C16); 5alpha-androstan-3alpha,17beta-diol monosulfate 1; pyruvate; urate; 1-methylguanosine; C-glycosyltryptophan; 1-eicosapentaenoylglycerophosphoethanolamine; 3-hydroxyoctanoate; oleoylcarnitine (C18); sphingosine 1-phosphate; phenylalanylalanine; alanine; 3-methylglutaroylcarnitine (C6); N-acetylcarnosine; isoleucine; dihydroferulic acid; homovanillate sulfate; uridine; 4-hydroxyhippurate; leucine; glycylproline; trimethylamine N-oxide; laurylcarnitine (C12); propionylglycine (C3); propionylcarnitine (C3); fumarate; L-urobilin; glycerate; gamma-glutamyllysine; myo-inositol; pregnen-diol disulfate; 5-hydroxyindoleacetate; 2-hydroxypalmitate; 3-methyl-2-oxobutyrate; N2,N5-diacetylornithine; 4-androsten-3beta,17beta-diol monosulfate 2; taurine; valylvaline; gamma-glutamyl glutamate; 3-carboxy-4-methyl-5-propyl-2-furanpropanoate (CMPF); carnitine; salicylate; succinate; isoleucylphenylalanine; riboflavin (Vitamin B2); pyrophosphate (PPi).

In a preferred embodiment, this set of biomarkers is constituted of at least two metabolites selected from sarcosine (N-methylglycine); HWESASLLR; iminodiacetate (IDA); 3-[3-(sulfooxy)phenyl]propanoic acid; leucylglycine; tetradecanedioate (C14); 3-hydroxybutyrate (BHBA); hexadecanedioate (C16); 3-dehydrocarnitine; caprate (10:0); threonylleucine; leucylglutamate; leucylalanine; N-oleoyltaurine; 2-hydroxybutyrate (AHB); a mix of 13-HODE and 9-HODE; sphinganine; hypoxanthine; glycolate (hydroxyacetate); taurocholenate sulfate; phenylacetate; myristate (14:0); margarate (17:0); valylglutamine; stearate (18:0); N-palmitoyltaurine; hydroxybutyrylcarnitine; glycerol; gamma-glutamylalanine; piperine.

In another preferred embodiment, the set of biomarkers is constituted of at least three metabolites selected from sarcosine (N-methylglycine); HWESASLLR; iminodiacetate (IDA); 3-[3-(sulfooxy)phenyl]propanoic acid; leucylglycine; tetradecanedioate (C14); 3-hydroxybutyrate (BHBA); hexadecanedioate (C16); 3-dehydrocarnitine; caprate (10:0); threonylleucine; leucylglutamate; leucylalanine; N-oleoyltaurine; 2-hydroxybutyrate (AHB); a mix of 13-HODE and 9-HODE; sphinganine; hypoxanthine; glycolate (hydroxyacetate); taurocholenate sulfate; phenylacetate; myristate (14:0); margarate (17:0); valylglutamine; stearate (18:0); N-palmitoyltaurine; hydroxybutyrylcarnitine; glycerol; gamma-glutamylalanine; piperine.

In a more preferred embodiment, the set of biomarkers is constituted of (i) at least one biomarker(s) selected from HWESASLLR and sarcosine (N-methylglycine), and (ii) at least one distinct biomarker(s) selected from HWESASLLR; iminodiacetate (IDA); 3-[3-(sulfooxy)phenyl]propanoic acid; sarcosine (N methylglycine); leucylglycine; tetradecanedioate (C14); 3-hydroxybutyrate (BHBA); hexadecanedioate (C16); 3-dehydrocarnitine; caprate (10:0); threonylleucine; leucylglutamate; leucylalanine; N-oleoyltaurine; 2-hydroxybutyrate (AHB); a mix of 13-HODE and 9-HODE; sphinganine; hypoxanthine; glycolate (hydroxyacetate); taurocholenate sulfate; phenylacetate; myristate (14:0); margarate (17:0); valylglutamine; stearate (18:0); N-palmitoyltaurine; hydroxybutyrylcarnitine; glycerol; gamma-glutamylalanine; piperine; laurate (12:0); 10-nonadecenoate (19:1n9); dihomolinoleate (20:2n6); eicosenoate (20:1n9 or 1n11); lysine; 3-hydroxydecanoate; palmitate (16:0); 3-hydroxyhippurate; 5-dodecenoate (12:1n7); acetylcarnitine (C2); 5 alpha-androstan-3beta,17beta-diol monosulfate 2; methyl-beta-glucopyranoside; 1-eicosapentaenoylglycerophosphocholine (20:5n3); docosadienoate (22:2n6); gamma-glutamylmethionine; methylpalmitate (15 or 2); pentadecanoate (15:0); docosatrienoate (22:3n3); myristoylcarnitine; linoleate (18:2n6); 1-stearoylglycerol (18:0); myristoleate (14:1n5); oleate (18:1n9); 13-methylmyristic acid; nonadecanoate (19:0); 10-heptadecenoate (17:1n7); 5alpha-androstan-3,17-diol monosulfate (alpha,beta or beta, alpha); 17-methylstearate; vaccenate (18:1n7); palmitoleate (16:1n7); adrenate (22:4n6); linolenate (18:3n3 or 3n6); docosapentaenoate (DPA; 22:5n3); dihomolinolenate (20:3n3 or 3n6), wherein an alteration of said presence, quantity, frequency or form is indicative of the presence, risk, subtype, progression or severity of said disease.

In a particularly preferred embodiment, said set of biomarkers contains at least HWESASLLR.

In another preferred embodiment, said set of biomarkers contains at least sarcosine (N-methylglycine).

In a more preferred embodiment, the set of biomarkers comprises at least iminodiacetate (IDA).

In another particularly preferred embodiment, said set of biomarkers contains at least 3-[3-(sulfooxy)phenyl]propanoic acid.

In a further preferred embodiment, the set of biomarkers comprises HWESASLLR used in combination with a metabolite selected from glutaroylcarnitine (C5); glycerate; threonylleucine; cysteine-glutathione disulfide; hypoxanthine; valylvaline; palmitate (16:0); sphinganine; sarcosine (N-methylglycine); homovanillate sulfate; leucylglycine; docosatrienoate (22:3n3); a mix of 13-HODE and 9-HODE; palmitoyl ethanolamide; acetylcarnitine (C2); taurocholenate sulfate; riboflavin (Vitamin B2); uridine; pregnen-diol di sulfate; 1-eicosapentaenoylglycerophosphocholine (20:5n3); stearate (18:0); hydroxybutyrylcarnitine; lysine; dihomolinoleate (20:2n6); dihomolinolenate (20:3n3 or 3n6); linoleate (18:2n6); gamma-glutamylalanine; leucylalanine; glycylproline; oleate (18:1n9); gamma-glutamyllysine; iminodiacetate (IDA); succinate; leucylglutamate; isoleucylphenylalanine; linolenate (18:3n3 or 3n6); glycolate (hydroxyacetate); salicylate; adenine; isoleucine; methionine; 6-oxopiperidine-2-carboxylic acid; gamma-glutamylmethionine; histidine; pyrophosphate (PPi); myo-inositol; 10-heptadecenoate (17:1n7); suberate (octanedioate); glycerol; 1-eicosapentaenoylglycerophosphoethanolamine; 3-dehydrocarnitine; sphingosine 1-phosphate; 5alpha-androstan-3,17-diol monosulfate (alpha,beta or beta, alpha); docosadienoate (22:2n6); 1-methylguanosine; 1-stearoylglycerol (18:0); eicosenoate (20:1n9 or 1n11); dihydroferulic acid; 3-methylglutaroylcarnitine (C6); trimethylamine N-oxide; alanine; urate; pentadecanoate (15:0); 10-nonadecenoate (19:1n9); 5alpha-androstan-3alpha,17beta-diol monosulfate 1; fumarate; docosapentaenoate (DPA; 22:5n3); palmitoleate (16:1n7); vaccenate (18:1n7); leucine; methylpalmitate (15 or 2); propionylcarnitine (C3); 3-hydroxydecanoate; 3-[3-(sulfooxy)phenyl]propanoic acid; piperine; 3-carboxy-4-methyl-5-propyl-2-furanpropanoate (CMPF); 2-hydroxypalmitate; ornithine; 3-hydroxybutyrate (BHBA); N2,N5-diacetylornithine; myristate (14:0); 4-androsten-3beta,17beta-diol monosulfate 2; taurolithocholate 3-sulfate; laurylcarnitine (C12); N-palmitoyltaurine; L-urobilin; ergothioneine; gamma-glutamylglutamate; laurate (12:0); margarate (17:0); palmitoylcarnitine (C16); oleoylcarnitine (C18); 5alpha-androstan-3beta,17beta-diol monosulfate 2; valylglutamine; 5-dodecenoate (12:1n7); pyruvate; caprate (10:0); 2-hydroxybutyrate (AHB); caprylate (8:0); 17-methyl stearate; phenylacetate; adrenate (22:4n6); nonadecanoate (19:0); tetradecanedioate (C14); N-acetylcarnosine; methyl-beta-glucopyranoside; citrate; N-acetylglycine; hexadecanedioate (C16); propionylglycine (C3).

In another preferred embodiment, the set of biomarkers comprises sarcosine (N-methylglycine) used in combination with a metabolite selected from N-oleoyltaurine; gamma-glutamylmethionine; margarate (17:0); linoleate (18:2n6); suberate (octanedioate); 10-nonadecenoate (19:1n9); docosadienoate (22:2n6); 13-methylmyristic acid; eicosenoate (20:1n9 or 1n11); methionine; myristoleate (14:1n5); tetradecanedioate (C14); 17-methylstearate; oleate (18:1n9); dihomolinoleate (20:2n6); methylpalmitate (15 or 2); gamma-glutamylalanine; gamma-glutamylglutamate; palmitoylcarnitine (C16); palmitoleate (16:1n7); 10-heptadecenoate (17:1n7); isoleucine; phenylacetate; adrenate (22:4n6); 6-oxopiperidine-2-carboxylic acid.

In a particular embodiment, the set of biomarkers comprises at least one combination selected from:
gamma-glutamyllysine and iminodiacetate (IDA),
hypoxanthine and tetradecanedioate (C14),
gamma-glutamylmethionine and iminodiacetate (IDA),
iminodiacetate (IDA) and isoleucine, and
3-[3-(sulfooxy)phenyl]propanoic acid and myo-inositol.

In an embodiment, the set of biomarkers comprises HWESASLLR, glutaroylcarnitine (C5) and methionine.

In a particular embodiment, HWESASLLR concentration is decreased from about 1 to 50%, preferably from about 5% to 25%, and more preferably of about 16%, in diseased subjects as compared to a concentration level in a control sample or in a reference situation.

In a particular embodiment, sarcosine (N-methylglycine) concentration is increased from about 1 to 50%, preferably from about 1% to 10%, and more preferably of about 3%, in diseased subjects as compared to a concentration level in a control sample or in a reference situation.

In a particular embodiment, 3-[3-(sulfooxy)phenyl]propanoic acid concentration is decreased from about 1 to 50%, preferably from about 5% to 25%, and more preferably of about 10%, in diseased subjects as compared to a concentration level in a control sample or in a reference situation.

In a particular embodiment, iminodiacetate (IDA) concentration is increased from about 1 to 50%, preferably from about 1% to 10%, and more preferably of about 5%, in diseased subjects as compared to a concentration level in a control sample or in a reference situation.

In another embodiment, the set of biomarkers comprises sarcosine (N-methylglycine) and HWESASLLR.

In another preferred embodiment, the set of biomarkers is constituted of at least two compounds selected from sarcosine (N-methylglycine); HWESASLLR; iminodiacetate (IDA); 3-[3-(sulfooxy)phenyl]propanoic acid; leucylglycine; tetradecanedioate (C14); 3-hydroxybutyrate (BHBA); hexadecanedioate (C16); 3-dehydrocarnitine; caprate (10:0); threonylleucine; leucylglutamate.

Preferred sets of biomarkers are selected from sets comprising:
glutaroylcarnitine (C5) and HWESASLLR,
glycerate and HWESASLLR,
HWESASLLR and threonylleucine,
cysteine-glutathione disulfide and HWESASLLR,
HWESASLLR and hypoxanthine,
HWESASLLR and valylvaline,
HWESASLLR and palmitate (16:0),
HWESASLLR and sphinganine,
HWESASLLR and sarcosine (N-methylglycine),
homovanillate sulfate and HWESASLLR,
HWESASLLR and leucylglycine,
docosatrienoate (22:3n3) and HWESASLLR,
a mix of 13-HODE and 9-HODE and HWESASLLR,
HWESASLLR and palmitoyl ethanolamide,
acetylcarnitine (C2) and HWESASLLR,
HWESASLLR and taurocholenate sulfate,
HWESASLLR and riboflavin (Vitamin B2),
HWESASLLR and uridine,
HWESASLLR and pregnen-diol disulfate,
1-eicosapentaenoylglycerophosphocholine (20:5n3) and HWESASLLR,
HWESASLLR and stearate (18:0),
HWESASLLR and hydroxybutyrylcarnitine,
HWESASLLR and lysine,
dihomolinoleate (20:2n6) and HWESASLLR,
dihomolinolenate (20:3n3 or 3n6) and HWESASLLR,
HWESASLLR and linoleate (18:2n6),
gamma-glutamylalanine and HWESASLLR,
HWESASLLR and leucylalanine,
glycylproline and HWESASLLR,
HWESASLLR and oleate (18:1n9),
gamma-glutamyllysine and HWESASLLR,
HWESASLLR and iminodiacetate (IDA),
HWESASLLR and succinate,
HWESASLLR and leucylglutamate,
HWESASLLR and isoleucylphenylalanine,
HWESASLLR and linolenate (18:3n3 or 3n6),
glycolate (hydroxyacetate) and HWESASLLR,
HWESASLLR and salicylate,
adenine and HWESASLLR,
HWESASLLR and isoleucine,
HWESASLLR and methionine,
6-oxopiperidine-2-carboxylic acid and HWESASLLR,
gamma-glutamylmethionine and HWESASLLR,
histidine and HWESASLLR,
HWESASLLR and pyrophosphate (PPi),
HWESASLLR and myo-inositol,
10-heptadecenoate (17:1n7) and HWESASLLR,
HWESASLLR and suberate (octanedioate),
glycerol and HWESASLLR,
1-eicosapentaenoylglycerophosphoethanolamine and HWESASLLR,
3-dehydrocarnitine and HWESASLLR,
HWESASLLR and sphingosine 1-phosphate,
5alpha-androstan-3,17-diol monosulfate (alpha,beta or beta,alpha) and HWESASLLR,
docosadienoate (22:2n6) and HWESASLLR,
N-oleoyltaurine and sarcosine (N-methylglycine),
1-methylguanosine and HWESASLLR,
1-stearoylglycerol (18:0) and HWESASLLR,
eicosenoate (20:1n9 or 1n11) and HWESASLLR,
dihydroferulic acid and HWESASLLR,
3-methylglutaroylcarnitine (C6) and HWESASLLR,
HWESASLLR and trimethylamine N-oxide,
alanine and HWESASLLR,
HWESASLLR and urate,
HWESASLLR and pentadecanoate (15:0),
10-nonadecenoate (19:1n9) and HWESASLLR,
5alpha-androstan-3alpha,17beta-diol monosulfate 1 and HWESASLLR,
fumarate and HWESASLLR,
docosapentaenoate (DPA; 22:5n3) and HWESASLLR,
HWESASLLR and palmitoleate (16:1n7),
HWESASLLR and vaccenate (18:1n7),
HWESASLLR and leucine,
HWESASLLR and methylpalmitate (15 or 2),
HWESASLLR and propionylcarnitine (C3),
3-hydroxydecanoate and HWESASLLR,
3-[3-(sulfooxy)phenyl]propanoic acid and HWESASLLR,
HWESASLLR and piperine,
3-carboxy-4-methyl-5-propyl-2-furanpropanoate (CMPF) and HWESASLLR,
2-hydroxypalmitate and HWESASLLR,
HWESASLLR and ornithine,
3-hydroxybutyrate (BHBA) and HWESASLLR,
HWESASLLR and N2,N5-diacetylornithine,
HWESASLLR and myristate (14:0),
4-androsten-3beta,17beta-diol monosulfate 2 and HWESASLLR,
HWESASLLR and taurolithocholate 3-sulfate,
HWESASLLR and laurylcarnitine (C12),
HWESASLLR and N-palmitoyltaurine,
HWESASLLR and L-urobilin,
ergothioneine and HWESASLLR,
gamma-glutamylglutamate and HWESASLLR,
HWESASLLR and laurate (12:0),
HWESASLLR and margarate (17:0),
HWESASLLR and palmitoylcarnitine (C16),
HWESASLLR and oleoylcarnitine (C18),
5alpha-androstan-3beta,17beta-diol monosulfate 2 and HWESASLLR,
HWESASLLR and valylglutamine,
5-dodecenoate (12:1n7) and HWESASLLR,
HWESASLLR and pyruvate,
caprate (10:0) and HWESASLLR,
2-hydroxybutyrate (AHB) and HWESASLLR,
caprylate (8:0) and HWESASLLR,
17-methylstearate and HWESASLLR,
HWESASLLR and phenylacetate,
adrenate (22:4n6) and HWESASLLR,
HWESASLLR and nonadecanoate (19:0),
HWESASLLR and tetradecanedioate (C14),
HWESASLLR and N-acetylcarnosine,
HWESASLLR and methyl-beta-glucopyranoside,
citrate and HWESASLLR,
HWESASLLR and N-acetylglycine,
hexadecanedioate (C16) and HWESASLLR,
HWESASLLR and propionylglycine (C3),
gamma-glutamylmethionine and sarcosine (N-methylglycine),
gamma-glutamyllysine and iminodiacetate (IDA),
margarate (17:0) and sarcosine (N-methylglycine),
linoleate (18:2n6) and sarcosine (N-methylglycine),
gamma-glutamylmethionine and iminodiacetate (IDA),
sarcosine (N-methylglycine) and suberate (octanedioate),
hypoxanthine and tetradecanedioate (C14),
10-nonadecenoate (19:1n9) and sarcosine (N-methylglycine),
docosadienoate (22:2n6) and sarcosine (N-methylglycine),
13-methylmyristic acid and sarcosine (N-methylglycine),
eicosenoate (20:1n9 or 1n11) and sarcosine (N-methylglycine),
methionine and sarcosine (N-methylglycine),
myristoleate (14:1n5) and sarcosine (N-methylglycine),
sarcosine (N-methylglycine) and tetradecanedioate (C14),
17-methylstearate and sarcosine (N-methylglycine),
oleate (18:1n9) and sarcosine (N-methylglycine),
dihomolinoleate (20:2n6) and sarcosine (N-methylglycine),
methylpalmitate (15 or 2) and sarcosine (N-methylglycine),
iminodiacetate (IDA) and isoleucine,
gamma-glutamylalanine and sarcosine (N-methylglycine),
gamma-glutamylglutamate and sarcosine (N-methylglycine),
palmitoylcarnitine (C16) and sarcosine (N-methylglycine),
palmitoleate (16:1n7) and sarcosine (N-methylglycine),
10-heptadecenoate (17:1n7) and sarcosine (N-methylglycine),
isoleucine and sarcosine (N-methylglycine),
3-[3-(sulfooxy)phenyl]propanoic acid and myo-inositol,
phenylacetate and sarcosine (N-methylglycine),
adrenate (22:4n6) and sarcosine (N-methylglycine),
6-oxopiperidine-2-carboxylic acid and sarcosine (N-methylglycine),
glutaroylcarnitine (C5) and HWESASLLR and phenylalanylalanine,
glutaroylcarnitine (C5) and HWESASLLR and isoleucylleucine,
glutaroylcarnitine (C5) and HWESASLLR and myristoylcarnitine,
glutaroylcarnitine (C5) and HWESASLLR and octadecanedioate (C18),
carnitine and glutaroylcarnitine (C5) and HWESASLLR,
asparagylleucine and glutaroylcarnitine (C5) and HWESASLLR,
3-hydroxyoctanoate and glutaroylcarnitine (C5) and HWESASLLR,
glutaroylcarnitine (C5) and HWESASLLR and taurine,
5-hydroxyindoleacetate and glutaroylcarnitine (C5) and HWESASLLR,
3-hydroxysebacate and glutaroylcarnitine (C5) and HWESASLLR,
3-hydroxysebacate and cysteine-glutathione disulfide and HWESASLLR,
HWESASLLR and myristoylcarnitine and sarcosine (N-methylglycine),
cysteine-glutathione disulfide and HWESASLLR and isoleucylleucine,
cysteine-glutathione disulfide and HWESASLLR and taurine,
asparagylleucine and cysteine-glutathione disulfide and HWESASLLR,
3-methyl-2-oxobutyrate and HWESASLLR and sphinganine,
cysteine-glutathione disulfide and HWESASLLR and octadecanedioate (C18),
HWESASLLR and phenylalanylalanine and sarcosine (N-methylglycine),
3-methyl-2-oxobutyrate and HWESASLLR and threonylleucine,
3-hydroxyoctanoate and HWESASLLR and threonylleucine,
5-hydroxyindoleacetate and HWESASLLR and sarcosine (N-methylglycine),
carnitine and HWESASLLR and threonylleucine,
3-methyl-2-oxobutyrate and 4-methylcatechol sulfate and HWESASLLR, and
13-methylmyristic acid and 4-methylcatechol sulfate and sarcosine (N-methylglycine).

Primary fatty acid amides (PFAM) represent valuable biomarkers, comprising PFAM (22:1), PFAM (20:1), and PFAM (22:2). These PFAM have the formula NH₂—CO—R, with R being either i) in the case of PFAM (20:1), an alkene of 19 carbon atoms with one cis or trans double bond or ii) in the case of PFAM (22:1), an alkene of 21 carbon atoms with one cis or trans double bond or iii) in the case of PFAM (22:2), an alkene of 21 carbon atoms with two double bonds that are independently cis or trans. PFAM (20:1) designates one single isomer or a mix of PFAM (20:1) isomers, PFAM (22:1) designates one single isomer or a mix of PFAM (22:1) isomers, and PFAM (22:2) designates one single isomer or a mix of PFAM (22:2) isomers.

In a particular embodiment, the above disclosed biomarkers or sets thereof are combined with one or more metabolite(s) selected from PFAM (22:1), PFAM (20:1), PFAM (22:2), hippurate, tyrosine, tryptophan, undecanedioate, isovalerate (C5), 1-palmitoylglycerol (16:0), dodecanedioate (C12), sebacate (decanedioate) and inosine.

Accordingly, in a particular embodiment, sets of biomarkers are selected from:
HWESASLLR and PFAM (22:1),
HWESASLLR and PFAM (20:1),
HWESASLLR and PFAM (22:2),
sarcosine (N-methylglycine) and PFAM (22:1),
sarcosine (N-methylglycine) and PFAM (20:1),
sarcosine (N-methylglycine) and PFAM (22:2),
iminodiacetate (IDA) and PFAM (22:1),
iminodiacetate (IDA) and PFAM (20:1),
iminodiacetate (IDA) and PFAM (22:2),
3-[3-(sulfooxy)phenyl]propanoic acid and PFAM (22:1),
3-[3-(sulfooxy)phenyl]propanoic acid and PFAM (20:1),
3-[3-(sulfooxy)phenyl]propanoic acid and PFAM (22:2),
HWESASLLR and inosine,
HWESASLLR and tryptophan,
HWESASLLR and tyrosine,
hippurate and HWESASLLR,
HWESASLLR and isovalerate (C5),
1-palmitoylglycerol (16:0) and HWESASLLR,
dodecanedioate (C12) and HWESASLLR,
glutaroylcarnitine (C5) and HWESASLLR and sebacate (decanedioate),
glutaroylcarnitine (C5) and HWESASLLR and undecanedioate,
cysteine-glutathione disulfide and HWESASLLR and sebacate (decanedioate),
HWESASLLR and threonylleucine and undecanedioate,
HWESASLLR and N-acetyltryptophan and tryptophan,
HWESASLLR and N-acetyltryptophan and tyrosine,
C-glycosyltryptophan and HWESASLLR and inosine,
3-hydroxyhippurate and HWESASLLR and inosine,
HWESASLLR and inosine and N-acetyltyrosine,
C-glycosyltryptophan and HWESASLLR and tryptophan,
4-hydroxyhippurate and HWESASLLR and inosine,
HWESASLLR and N-acetyltyrosine and tryptophan,
4-hydroxyhippurate and HWESASLLR and sebacate (decanedioate), and
1-palmitoylglycerol (16:0)+3-hydroxyhippurate+HWESASLLR.

In a particular embodiment, diagnosing AD and related disorders, comprises the identification, within LC/MS or GC/MS mass profile from sample of the mammal, of a metabolite mass profile determined as specific for AD or a related disorder, said profile being constituted by 2, 3, 4 or 5 mass peaks corresponding to the dominant ions of the metabolites identified in Tables 1 and 2.

In a particular embodiment, any of the above biomarkers or their combinations are used in a method of diagnosing AD or related disorders, in conjunction with at least one additional diagnostic test or biomarker for AD or related disorders, selected preferably from nucleic acids, proteins, metabolites, neurophysiological (e.g. electroencephalography), genetic, brain imaging, clinical and cognitive test or biomarker. Such diagnostic test or biomarker can be done or measured concomitantly, before, or after the measure of biomarkers of the invention. Said additional diagnostic biomarkers can be detected in any sample convenient for the assay.

Said additional protein biomarker, which can be used for diagnosing AD or related disorders, can be selected from proteins listed in WO2011/012672. Other candidates as proteinaceous biomarkers known in the art as an aid in diagnosing AD are Aβ₄₂, Tau or P-Tau₁₈₁, which can be dosed from the LCR. A decreased in Aβ₄₂, and an increase of Tau and P-Tau₁₈₁ are noticed in the LCR of AD patients. When talking about plasmatic biomarkers, the usefulness of Aβ peptides is at least controversial [17], but Aβ₄₂/Aβ₄₀ ratio seems to be of some use as a low Aβ₄₂/Aβ₄₀ plasmatic ratio has been associated with the risk of a more rapid cognitive decline [17].

Consequently, in an embodiment, any of the biomarkers of the invention or their combinations are used in a method of diagnosing AD or related disorders, in conjunction with the measure of the determination of Aβ₄₂, Tau and/or P-Tau₁₈₁ in the LCR.

In another embodiment any of the biomarkers of the invention or their combinations are used in a method of diagnosing AD or related disorders or the risk of a rapid cognitive decline, in conjunction with the measure of plasmatic Aβ₄₂/Aβ₄₀ ratio.

Brain imaging tests that can be implemented in conjunction with any of the biomarkers of the invention can be for example:
detection and quantitation tests of Aβ deposition and/or fibrillar Aβ burden in brain, or of pattern of deposition thereof, by imaging methods as positron emission tomography, which can be indicative of AD or of AD evolution,
morphologic brain imaging, for instance measure of the volume of the hippocampus, which can be indicative of AD or of AD evolution.

In a more particular embodiment, biomarkers of the invention are used to diagnose AD or a related disorder in patient(s) identified as being at risk of developing AD or suspected of suffering from prodromal AD. For instance such patient(s) can have been diagnosed bearing ApoE c4 allele of ApoE.

Biomarkers of the invention can also be used in addition of any cognitive test used to assess the cognitive status of a patient. Such tests are, for example, Mini-Mental State Examination (MMSE), Modified Mini-Mental State Examination (3MS), Abbreviated Mental Test Score (AMTS), Dementia questionnaire for persons with Mental Retardation (DMR), Cognitive Abilities Screening Instrument (CASI), Trail-making test, Clock drawing test, Alzheimer's disease assessment scale-Cognition (ADAS-Cog), General Practitioner Assessment of Cognition (GPCOG), Montreal Cognitive Assessment (MoCA), or Rowland Universal Dementia Assessment Scale (RUDAS).

In a preferred embodiment, any of the biomarkers of the invention is used in conjunction with MMSE.

In another preferred embodiment, biomarkers of the invention are used to diagnose AD or a related disorder in patient(s) identified as being at risk of developing AD or suspected of suffering from prodromal AD because of the result they obtained in the MMSE. As pointed out above, the MMSE scores are affected by the age and the cultural level of the subject. Thus these scores must be corrected in function of these criteria before their interpretation. As an indicative basis, according to the Consortium to Establish a Registry for Alzheimer's Disease (CERAD), a score comprised between 19 and 24 is associated with a weak dementia, between 10 and 18 with a moderate dementia and finally, a score under 10 corresponds to a severe dementia.

Another aspect of the invention relates to the use of one or more biomarker(s) selected from biomarkers disclosed herein in a method of AD diagnosis in a mammalian subject.

The method of the invention is applicable to any biological sample of the mammal to be tested. Examples of such samples include blood, plasma, serum, saliva, urine, ascites, sputum, aerosols, sweat or the like. Level of metabolites derived therefrom can also be measured from tissue biopsies or feces. The sample can be obtained by any technique known per se in the art, for example by collection using e.g., non-invasive techniques, or from collections or banks of samples, etc. The sample can in addition be pretreated to facilitate the accessibility of the target biomarker, to allow the dosage of said biomarker by a dedicated method (e.g. derivatization of amino acids to allow their subsequent dosage by spectrophotometry), or to enrich for the target biomarker, for example by lysis (mechanical, chemical, enzymatic, etc.), purification, extraction, centrifugation, separation, precipitation, etc. Serum preparation from blood can be performed as exemplified in experimental section. Several other sample preparations can be used such as liquid-liquid extraction, protein precipitation and solid-phase extraction [18].

In a preferred embodiment, levels of biomarkers of the invention are determined from blood, plasma, serum, saliva, or urine sample(s).

In another embodiment, biomarker(s) may be quantified from different samples from the same mammal.

The invention is applicable to any mammal, preferably to a human.

In an embodiment, said human is not yet suffering from a significant cognitive impairment when compared with people of same age and cultural level.

In another embodiment, said human presents Aβ aggregates deposition or a fibrillar Aβ burden in brain, associated or not with a cognitive impairment.

It is known that patients with Down's syndrome exhibit an extremely high incidence of early onset of AD [19]. Consequently, in another embodiment, said human is suffering from Down's syndrome.

The levels of said biomarker(s) may be determined by any method known per se in the art, such as, without limitation, immunological methods, biochemical methods, chromatographic methods, enzymatic methods, cell based assays, in vitro tests, LC/MS, GC/MS etc. Such assays are routine and well known in the art. The levels of biomarker(s) determined may be compared to a reference value, a control, or a mean value, wherein a deviation from said value is indicative of the presence, risk, progression and/or severity of AD or related disorders. The deviation should typically be superior to 1%, preferably superior to 2%, more preferably superior to 2.5%, even more preferably superior to 5%. In other embodiments, deviation may be of about 10%, 15%, 20%, 30%, 40%, 50%, 60%, 70%, 80%, 90% or 100%.

In another embodiment, differential presence of other metabolites related to the same metabolic pathways than the biomarkers of the invention is quantified.

In still another aspect, the present invention provides a kit comprising a solid support comprising at least one capture agent attached thereto, wherein said at least one capture agent binds or reacts with one biomarker of the present invention. Typically, the kit may comprise several distinct capture agents which bind to a distinct biomarker. The at least one binding agent is preferably selective for a biomarker, such as an antibody or a derivative thereof, an aptamer, etc.

In a preferred embodiment, the kit of the invention comprises a solid support comprising at least one capture agent attached thereto (for instance an antibody or an aptamer), wherein the capture agent binds or reacts with one biomarker from the biomarkers disclosed herein. In a preferred embodiment, the kit of the invention comprises at least one compound binding to or reacting with at least one biomarker selected from the biomarkers disclosed herein for the diagnostic, prognostic and/or for assessing the efficacy of a treatment or following the evolution of AD or related disorders.

In addition to LC/MS method for assaying biomarkers of the invention, other assays exist as discussed below in an illustrative way.

Amino acids (or derivative thereof) quantification

### HPLC-Spectrophotometry Whole Amino Acids Profile.

Amino acids blood tests are well known in the art. They are, for example, commonly used to determine aminogram of young children in order to diagnose aminoacidopathies.

HPLC/spectrophotometry methods are the most commonly used methods for assaying whole amino acids (or their derivatives) at once from biological fluids. They are more often automatized. Amino acids need to be derivatized to be detectable by absorbance spectrophotometry. Derivatization can be performed before or after HPLC amino acids separation.

Derivatization consists in the covalently linking of amino acids to a chromophoric moiety thereby rendering modified amino acids easily detectable by UV, visible or fluorometric spectrophotometry. Derivatization can be performed, for example, with Phenyl-Thio-Cyanate (PTC, UV spectrophotometry), Ortho-PhtAldehyde, (OPA; UV or fluorometric spectro-photometry), DimethylAmino-1-NaphtaleneSulfonYL (DANSYL; visible spectrophotometry), or 9-FluorenylMethOxyCarbonyl (FMOC; fluorometric spectrophotometry).

Protocol for amino acids quantization using OPA derivatization is extensively described in Babu et al. [20].

Commercial kits are also sold for performing HPLC assays to measure amino acids quantity in human fluids as for example “Phenylalanine, Tyrosine & Tryptophan HPLC Assay” from Eagle biosciences (Catalog Number: PNL31-H100).

### Kits Dedicated to the Quantification of Specific Amino Acids

Amino acids biomarkers of the invention can also be specifically quantified from biological samples using off the shelf dedicated detection and quantification kits.

Aspartic acid can be assayed using, for example, “Aspartate assay kit” (Biovision, ref K552-100): an enzymatic colorimetric assay based of the enzymatic conversion of aspartate in pyruvate. L-tryptophan can be measured using “Bridge-It® L-Tryptophan Fluorescence Assay” (Mediomics) which is based on the activity of tryptophan repressor protein and can detect tryptophan for instance in human urine or serum.

Fatty Acid Detection and Quantitation

Fatty acids of the invention and related compounds (i.e. dodecanedioic acid; sebacic acid; azelaic acid, caproic acid, undecanedioic acid, 9,12-dioxo-dodecanoic acid, nonenedioic acid, octadecadienoyl-glycero-3-phosphate) can also be identified by HPLC (reviewed by Lima and Abdalla, 2002, and Chen and Chuang, 2002) [21,22] or GC methods (see in Bondia-Pons et al. in 2004 [23] for example) well known by the man of the art. These methods usually need sample preparation steps as lipids extraction, purification and derivatization; they can be coupled or not to different detection and quantification methods, depending on the derivatization method that has been used. Reference compounds can easily be found to allow correct identification of the fatty acids which are searched for.

Immunological and Aptamers Based Methods

Immunological methods are methods that use an antibody to specifically bind an antigen (e.g. a biomarker, fragments and derivatives thereof.). The immunological method is used, in particular, to isolate, target, and/or quantify the antigen. For example, immunological methods include but are not limited to competitive and non-competitive assay systems using techniques such as western blots, radioimmunoassays, ELISA, “sandwich” immunoassays, immunoprecipitation assays, immunodiffusion assays, fluorescent immunoassays.

Antibody refers to a polypeptide ligand substantially encoded by an immunoglobulin gene or immunoglobulin genes, or fragments thereof, which specifically binds and recognizes an epitope (e.g. an antigen). The term “antibody”, as used herein, also includes antibody fragments either produced by the modification of whole antibodies or those synthetized de novo using recombinant DNA methodologies. It also includes polyclonal antibodies, monoclonal antibodies, chimeric antibodies, humanized antibodies or single chain antibodies.

Detection methods for assaying metabolites of the invention could use an aptamer that specifically binds to the searched metabolites. Aptamers are synthetic ssDNA or RNA molecules that recognize a ligand with a high specificity and affinity; they can represent a valuable alternative to antibodies in the case of metabolites with no or a low immunogenicity. They can be used for assaying metabolites of any kind, and their specificity allows the differentiation of closely related molecules. They can be easily synthetized by selex technique and variations thereof which are well known in the art [24] or chosen from a commercial library as for instance that of Aptagen (www.aptagen.com). Detection or quantification is performed somewhat in the same way that for well-known immunological methods or with dedicated methods[25].

Further aspects and advantages of this invention will be disclosed in the following experimental section, which shall be considered as illustrative only.

### EXAMPLES

### A) Metabolome Analysis

### 1. Sample Preparation

### 1.1. Human Plasma Samples

Plasmas from 28 healthy control subjects and 28 Alzheimer's disease (AD) patients have been collected (Table 3). AD samples came from Department of Neurology, Memory Research Resources Center (Montpellier University Hospital Gui de Chauliac, France) and plasma samples of age-matched controls were collected by Institut de Sante Publique d'Epidémiologie et de Développement (ISPED, University of Bordeaux, France).

TABLE 3

| Data | AD (n = 28) | CTRL (n = 28) |
|---|---|---|
| Gender (F/M) | 13/15 | 20/8 |
| Age | 76.81 and/−5.78 | 78.88 and/−1.93 |
| MMSE | 18 and/−4.26 | N/A |

### 1.2. Sample Processing

After thawing of the deep-frozen samples at room temperature, a step of protein precipitation with methanol was performed to remove protein while allowing maximum recovery of small molecules. The resulting extract was divided into four fractions; two for analysis by LC and one for analysis by GC, and one spare. Samples were placed briefly on a TurboVap® (Zymark) to remove the organic solvent. Each sample was dried under vacuum. Samples were then prepared for the appropriate instrument, either LC/MS or GC/MS. The samples were run in a randomized fashion.

### 2. Acquisition of the Metabolic Profiles

### 2.1. LC-MS/MS

The LC/MS was performed on a Waters ACQUITY ultra-performance liquid chromatography (UPLC) and a ThermoFisher Scientific Orbitrap Elite high resolution/accurate mass spectrometer, which consisted of a heated electrospray ionization (HESI) source and Orbitrap mass analyzer operated at 30,000 mass resolution. The sample extract was dried then reconstituted in acidic or basic LCcompatible solvents, each of which contained 8 or more injection standards at fixed concentrations to ensure injection and chromatographic consistency. One aliquot was analyzed using acidic positive ion optimized conditions and the other using basic negative ion optimized conditions in two independent injections using separate dedicated columns. Extracts reconstituted in acidic conditions were gradient eluted using water and methanol containing 0.1% formic acid, while the basic extracts, which also used water/methanol, contained 6.5 mM Ammonium Bicarbonate. The MS analysis alternated between MS and data-dependent MS2 scans using dynamic exclusion.

### 2.2. GC/MS.

The samples destined for GC/MS analysis were re-dried under vacuum desiccation for a minimum of 24 hours prior to being derivatized under dried nitrogen using bistrimethyl-silyl-triflouroacetamide (BSTFA). The GC column was 5% phenyl and the temperature ramp was from 40° to 300° C. in a 16 minute period. Samples were analyzed on a Thermo-Finnigan

Trace DSQ fast-scanning single-quadrupole mass spectrometer using electron impact ionization. The instrument was tuned and calibrated for mass resolution and mass accuracy on a daily basis.

3. LC/MS and/or GC/MS Raw Data Treatment.

The data extraction of the raw mass spec data files yielded information that was loaded into a relational database. Once in the database the information was examined and appropriate QC limits were imposed and peaks are identified.

### 4. Biomarker Identification.

The annotations of the raw variables according to the mass m/z of each signal were performed by comparison to library entries of purified standards or recurrent unknown entities. The combination of chromatographic properties and mass spectra gave an indication of a match to the specific compound or an isobaric entity. Additional entities could be identified by virtue of their recurrent nature (both chromatographic and mass spectral). A variety of curation procedures were carried out to ensure that a high quality data set was made available for statistical analysis and data interpretation. QC and curation processes were designed to ensure accurate and consistent identification of true chemical entities, and to remove those representing system artifacts, mis-assignments, and background noise.

B) Results Analysis-Identification of Biomarkers of Interest

### 1. Identification of Metabolites Discriminating Alzheimer's Disease Versus Control Samples

### 1.1 General Principles

Statistical analyses were performed using R version 3.1.2. A logarithmic transformation (log 10) was applied to metabolic variables in order to control the variances and for normality purpose. Statistical tests where two-tailed and conducted at the 5% significance level.

### 1.2 Comparison of Metabolite Levels Between AD and CTRL Samples

In order to identify metabolites that discriminate AD versus CTRL groups, a p-value of the differential level between AD and CTRL groups was computed using a linear model (lm( ) R function) adjusted on Age and Gender co-variables for each of the 866 variables (yᵢ) that passed the quality-control treatment:
yᵢ˜Status and Age and Gender

Analysis revealed that 220 metabolites (Table 4 below) were significantly different between plasma samples of AD patients and CTRL subjects. 129 of these metabolites were definitively identified by comparison to standard chemicals, whereas 11 were only annotated on the basis of public databases (Table 2).

### 1.3 Classification Performance of Identified Metabolites

Classification models from single or combination of variables are evaluated with AUC, Sensitivity and Specificity based on a Linear-Discriminant-Analysis (LDA) repeated random sub-sampling validation in order to avoid overfiting (caret R package [26]).

Partial Least Squares Discriminant Analysis (PLS-DA) has been used to perform a supervised classification where the most discriminant variables are determined by the ability to generate VIP scores [27] (DiscriMiner R package). These VIP scores estimate the importance of each predictor variable and are often used to select those predictors that are most influential in a given output response [28,29]. A variable with a VIP score close to or greater than one being considered important in given model [28,30].

VIP scores of biomarkers of the invention have been determined and are indicated in Table 4.

TABLE 4

| | | | | Variation in AD |
|---|---|---|---|---|
| | VIP | P- | | as compared to |
| Metabolite ranked as a function of their VIP score | score >1 | value | VIP | control |
| HWESASLLR | Yes | 0.0000 | 1.99 | decrease |
| Leucylalanine | Yes | 0.0000 | 1.53 | increase |
| iminodiacetate (IDA) | Yes | 0.0000 | 1.54 | increase |
| N-oleoyltaurine | Yes | 0.0000 | 1.36 | increase |
| Leucylglycine | Yes | 0.0000 | 1.32 | increase |
| tetradecanedioate (C14) | Yes | 0.0000 | 1.30 | increase |
| 3-hydroxybutyrate (BHBA) | Yes | 0.0000 | 1.30 | increase |
| sebacate (decanedioate) | Yes | 0.0000 | 1.31 | increase |
| hexadecanedioate (C16) | Yes | 0.0000 | 1.25 | increase |
| 3-dehydrocarnitine | Yes | 0.0000 | 1.24 | increase |
| 3-[3-(sulfooxy)phenyl]propanoic acid | Yes | 0.0000 | 1.35 | decrease |
| caprate (10:0) | Yes | 0.0001 | 1.18 | increase |
| 2-hydroxybutyrate (AHB) | Yes | 0.0001 | 1.15 | increase |
| threonylleucine | Yes | 0.0001 | 1.22 | increase |
| mix of 13-HODE and 9-HODE | Yes | 0.0001 | 1.13 | increase |
| sphinganine | Yes | 0.0002 | 1.10 | increase |
| leucylglutamate | Yes | 0.0002 | 1.13 | increase |
| hypoxanthine | Yes | 0.0003 | 1.10 | increase |
| glycolate (hydroxyacetate) | Yes | 0.0003 | 1.24 | increase |
| taurocholenate sulfate | Yes | 0.0004 | 1.10 | increase |
| phenylacetate | Yes | 0.0004 | 1.14 | increase |
| myristate (14:0) | Yes | 0.0005 | 1.25 | increase |
| margarate (17:0) | Yes | 0.0006 | 1.27 | increase |
| valylglutamine | Yes | 0.0006 | 1.07 | increase |
| stearate (18:0) | Yes | 0.0007 | 1.27 | increase |
| N-palmitoyltaurine | Yes | 0.0007 | 1.02 | increase |
| hydroxybutyrylcarnitine | Yes | 0.0008 | 1.02 | increase |
| glycerol | Yes | 0.0009 | 1.15 | increase |
| sarcosine (N-methylglycine) | Yes | 0.0009 | 1.27 | increase |
| gamma-glutamylalanine | Yes | 0.0010 | 1.12 | decrease |
| piperine | Yes | 0.0010 | 1.01 | decrease |
| laurate (12:0) | Yes | 0.0012 | 1.16 | increase |
| 10-nonadecenoate (19:1n9) | Yes | 0.0012 | 1.26 | increase |
| dihomolinoleate (20:2n6) | Yes | 0.0014 | 1.20 | increase |
| eicosenoate (20:1n9 or 1n11) | Yes | 0.0014 | 1.22 | increase |
| lysine | Yes | 0.0015 | 1.01 | decrease |
| 3-hydroxydecanoate | Yes | 0.0016 | 1.12 | increase |
| palmitate (16:0) | Yes | 0.0017 | 1.24 | increase |
| 3-hydroxyhippurate | Yes | 0.0017 | 1.09 | decrease |
| 5-dodecenoate (12:1n7) | Yes | 0.0018 | 1.15 | increase |
| acetylcarnitine (C2) | Yes | 0.0023 | 1.01 | increase |
| 5alpha-androstan-3beta,17beta-diol monosulfate 2 | Yes | 0.0023 | 1.01 | decrease |
| methyl-beta-glucopyranoside | Yes | 0.0024 | 1.01 | decrease |
| 1-eicosapentaenoylglycerophosphocholine (20:5n3) | Yes | 0.0025 | 1.11 | decrease |
| docosadienoate (22:2n6) | Yes | 0.0025 | 1.16 | increase |
| gamma-glutamylmethionine | Yes | 0.0030 | 1.03 | decrease |
| methylpalmitate (15 or 2) | Yes | 0.0031 | 1.18 | increase |
| pentadecanoate (15:0) | Yes | 0.0035 | 1.14 | increase |
| docosatrienoate (22:3n3) | Yes | 0.0036 | 1.06 | increase |
| myristoylcarnitine | Yes | 0.0036 | 1.07 | increase |
| linoleate (18:2n6) | Yes | 0.0044 | 1.16 | increase |
| hippurate | Yes | 0.0049 | 0.96 | decrease |
| 1-stearoylglycerol (18:0) | Yes | 0.0051 | 1.21 | increase |
| myristoleate (14:1n5) | Yes | 0.0052 | 1.13 | increase |
| oleate (18:1n9) | Yes | 0.0054 | 1.17 | increase |
| 13-methylmyristic acid | Yes | 0.0069 | 1.09 | increase |
| nonadecanoate (19:0) | Yes | 0.0115 | 1.06 | increase |
| 10-heptadecenoate (17:1n7) | Yes | 0.0125 | 1.17 | increase |
| 5alpha-androstan-3,17-diol monosulfate (alpha,beta or | Yes | 0.0159 | 1.02 | decrease |
| beta,alpha) | | | | |
| 17-methylstearate | Yes | 0.0183 | 1.07 | increase |
| vaccenate (18:1n7) | Yes | 0.0205 | 1.06 | increase |
| palmitoleate (16:1n7) | Yes | 0.0278 | 1.11 | increase |
| adrenate (22:4n6) | Yes | 0.0278 | 1.02 | increase |
| linolenate (18:3n3 or 3n6) | Yes | 0.0293 | 1.05 | increase |
| docosapentaenoate (DPA; 22:5n3) | Yes | 0.0325 | 1.13 | increase |
| dihomolinolenate (20:3n3 or 3n6) | Yes | 0.0406 | 1.09 | increase |
| dodecanedioate (C12) | No | 0.0013 | 0.97 | increase |
| adenine | No | 0.0023 | 0.97 | increase |
| 3-hydroxysebacate | No | 0.0027 | 0.92 | increase |
| octadecanedioate (C18) | No | 0.0027 | 0.91 | increase |
| isoleucylleucine | No | 0.0032 | 0.92 | increase |
| ergothioneine | No | 0.0036 | 0.93 | decrease |
| N-acetylglycine | No | 0.0044 | 0.99 | increase |
| caprylate (8:0) | No | 0.0045 | 0.88 | increase |
| tryptophan | No | 0.0055 | 0.89 | decrease |
| citrate | No | 0.0059 | 0.86 | increase |
| palmitoyl ethanolamide | No | 0.0063 | 0.88 | increase |
| histidine | No | 0.0064 | 0.84 | decrease |
| asparagylleucine | No | 0.0068 | 0.83 | increase |
| 4-methylcatechol sulfate | No | 0.0071 | 0.83 | decrease |
| N-acetyltyrosine | No | 0.0079 | 0.84 | decrease |
| suberate (octanedioate) | No | 0.0086 | 0.89 | increase |
| methionine | No | 0.0086 | 0.85 | decrease |
| cysteine-glutathione disulfide | No | 0.0103 | 0.86 | increase |
| 6-oxopiperidine-2-carboxylic acid | No | 0.0111 | 0.83 | increase |
| N-acetyltryptophan | No | 0.0138 | 0.77 | decrease |
| glutaroylcarnitine (C5) | No | 0.0141 | 0.79 | increase |
| taurolithocholate 3-sulfate | No | 0.0143 | 0.76 | increase |
| inosine | No | 0.0159 | 0.80 | increase |
| tyrosine | No | 0.0164 | 0.81 | decrease |
| ornithine | No | 0.0178 | 0.75 | decrease |
| palmitoylcarnitine (C16) | No | 0.0184 | 0.99 | increase |
| 5alpha-androstan-3alpha,17beta-diol monosulfate 1 | No | 0.0184 | 0.84 | decrease |
| pyruvate | No | 0.0184 | 0.77 | decrease |
| urate | No | 0.0189 | 0.85 | decrease |
| 1-methylguanosine | No | 0.0193 | 0.73 | increase |
| 1-eicosapentaenoylglycerophosphoethanolamine | No | 0.0211 | 0.75 | decrease |
| 3-hydroxyoctanoate | No | 0.0211 | 0.81 | increase |
| oleoylcarnitine (C18) | No | 0.0217 | 0.98 | increase |
| sphingosine 1-phosphate | No | 0.0224 | 0.80 | increase |
| phenylalanylalanine | No | 0.0237 | 0.82 | increase |
| C-glycosyltryptophan | No | 0.0239 | 0.71 | increase |
| alanine | No | 0.0251 | 0.74 | decrease |
| 3-methylglutaroylcarnitine (C6) | No | 0.0251 | 0.73 | increase |
| N-acetylcarnosine | No | 0.0254 | 0.81 | decrease |
| 1-palmitoylglycerol (16:0) | No | 0.0258 | 0.81 | increase |
| isoleucine | No | 0.0268 | 0.75 | decrease |
| dihydroferulic acid | No | 0.0269 | 0.71 | increase |
| homovanillate sulfate | No | 0.0276 | 0.79 | increase |
| uridine | No | 0.0278 | 0.84 | decrease |
| leucine | No | 0.0289 | 0.72 | decrease |
| glycylproline | No | 0.0305 | 0.68 | increase |
| 4-hydroxyhippurate | No | 0.0305 | 0.72 | decrease |
| trimethylamine N-oxide | No | 0.0307 | 0.72 | increase |
| laurylcarnitine (C12) | No | 0.0307 | 0.98 | increase |
| propionylglycine (C3) | No | 0.0309 | 0.76 | decrease |
| propionylcarnitine (C3) | No | 0.0313 | 0.78 | decrease |
| fumarate | No | 0.0328 | 0.82 | increase |
| L-urobilin | No | 0.0328 | 0.70 | increase |
| glycerate | No | 0.0332 | 0.67 | decrease |
| gamma-glutamyllysine | No | 0.0332 | 0.85 | decrease |
| myo-inositol | No | 0.0333 | 0.74 | increase |
| pregnen-diol disulfate | No | 0.0338 | 0.67 | increase |
| isovalerate (C5) | No | 0.0357 | 0.68 | increase |
| 5-hydroxyindoleacetate | No | 0.0373 | 0.92 | increase |
| undecanedioate | No | 0.0377 | 0.70 | increase |
| 2-hydroxypalmitate | No | 0.0380 | 0.88 | increase |
| 3-methyl-2-oxobutyrate | No | 0.0381 | 0.71 | increase |
| N2,N5-diacetylornithine | No | 0.0385 | 0.65 | decrease |
| 4-androsten-3beta,17beta-diol monosulfate 2 | No | 0.0393 | 0.83 | decrease |
| taurine | No | 0.0403 | 0.67 | increase |
| valylvaline | No | 0.0409 | 0.77 | increase |
| gamma-glutamylglutamate | No | 0.0423 | 0.95 | decrease |
| 3-carboxy-4-methyl-5-propyl-2-furanpropanoate (CMPF) | No | 0.0424 | 0.69 | decrease |
| carnitine | No | 0.0451 | 0.69 | decrease |
| salicylate | No | 0.0453 | 0.63 | decrease |
| succinate | No | 0.0460 | 0.70 | increase |
| isoleucylphenylalanine | No | 0.0467 | 0.63 | decrease |
| riboflavin (Vitamin B2) | No | 0.0484 | 0.76 | increase |
| pyrophosphate (PPi) | No | 0.0500 | 0.85 | increase |

### 1.4 Sets of Biomarkers of the Invention Allow an Accurate and Efficient Diagnostic of AD and Related Disorders.

Biomarkers or set of biomarkers are currently characterized by their AUC, sensitivity and specificity. The AUC give a global view on the efficiency of a given biomarker by representing the concordance of the diagnostic and disease state. The value of the AUC ranges from 0.5 (no discrimination) to 1.0 (perfect discrimination). Sensitivity is the proportion of subjects who are correctly categorized as having disease among those who truly have the disease. Similarly, specificity is the proportion of subjects who are correctly categorized as not having the disease among all subjects who truly don't have the disease.

In order identify efficient classifiers (biomarker or set of biomarkers) for the diagnostic of AD and related disorders, a linear discriminant analysis was performed [31]. AUC, sensitivity and specificity were computed as the mean of 1000 resampling iterations. For each iteration, ⅔ of the samples were used to train the classifier, and the remaining ⅓ were used to test the classifier and to provide AUC, sensitivity and specificity estimates.

Among all the identified single biomarkers, 13 stand out clearly, exhibiting a VIP>1, a p value<0.001 and a sensitivity >80% (Table 5).

TABLE 5

| | AUC | Sensibility | Sensitivity |
|---|---|---|---|
| Metabolite | (AD) | (AD) | (AD) |
| HWESASLLR | 95.3% | 89.4% | 82.8% |
| iminodiacetate (IDA) | 83.3% | 88.0% | 68.9% |
| tetradecanedioate (C14) | 79.1% | 89.2% | 69.9% |
| sarcosine (N-methylglycine) | 85.3% | 81.3% | 70.9% |
| 3-[3-(sulfooxy)phenyl]propanoic acid | 80.3% | 81.5% | 67.3% |
| 3-dehydrocarnitine | 76.3% | 83.1% | 68.3% |
| leucylglycine | 79.5% | 82.8% | 65.2% |
| sebacic acid | 79.2% | 81.5% | 66.4% |
| hexadecanedioate (C16) | 75.1% | 86.2% | 63.3% |
| leucylglutamate | 78.1% | 80.5% | 61.8% |
| threonylleucine | 77.1% | 85.1% | 57.8% |
| 3-hydroxybutyrate (BHBA) | 76.0% | 86.5% | 53.0% |
| caprate (10:0) | 77.0% | 82.8% | 55.0% |

Though biomarkers of the invention are particularly efficient for diagnosing AD and related disorders when used alone, the use of sets of at least two biomarkers is of interest in order to increase the sensitivity and/or the specificity of diagnostic tests.

Inventors have been able to select several sets of biomarkers of the invention with very satisfying sensitivity and specificity which are listed in Table 6. Noteworthy, all these sets exhibit a sensitivity and specificity superior to 80%. All of these sets exhibit an AUC above 0.84.

TABLE 6

| | AUC | Sensibility | Sensitivity |
|---|---|---|---|
| Metabolite combinations | (AD) | (AD) | (AD) |
| glutaroylcarnitine (C5) and HWESASLLR | 97.9% | 94.4% | 94.6% |
| glycerate and HWESASLLR | 96.8% | 93.7% | 88.1% |
| HWESASLLR and threonylleucine | 96.3% | 98.6% | 82.6% |
| cysteine-glutathione disulfide and HWESASLLR | 95.1% | 93.9% | 88.1% |
| HWESASLLR and hypoxanthine | 95.9% | 92.3% | 88.6% |
| HWESASLLR and valylvaline | 95.9% | 92.0% | 88.0% |
| HWESASLLR and palmitate (16:0) | 96.2% | 90.8% | 88.8% |
| HWESASLLR and sphinganine | 96.5% | 92.7% | 86.6% |
| HWESASLLR and sarcosine (N-methylglycine) | 94.5% | 93.0% | 88.1% |
| homovanillate sulfate and HWESASLLR | 96.6% | 91.8% | 86.8% |
| HWESASLLR and leucylglycine | 96.7% | 91.9% | 86.4% |
| docosatrienoate (22:3n3) and HWESASLLR | 96.4% | 88.8% | 89.7% |
| a mix of 13-HODE and 9-HODE, and HWESASLLR | 96.0% | 90.3% | 88.4% |
| HWESASLLR and palmitoyl ethanolamide | 95.0% | 90.8% | 88.3% |
| acetylcarnitine (C2) and HWESASLLR | 95.3% | 90.7% | 88.0% |
| HWESASLLR and taurocholenate sulfate | 95.9% | 90.9% | 87.0% |
| HWESASLLR and riboflavin (Vitamin B2) | 95.9% | 93.3% | 83.9% |
| HWESASLLR and uridine | 96.4% | 92.3% | 84.3% |
| HWESASLLR and pregnen-diol disulfate | 95.1% | 88.8% | 89.2% |
| 1-eicosapentaenoylglycerophosphocholine (20:5n3) and | 94.5% | 92.4% | 85.8% |
| HWESASLLR | | | |
| HWESASLLR and stearate (18:0) | 96.2% | 90.6% | 85.8% |
| HWESASLLR and hydroxybutyrylcarnitine | 94.9% | 91.1% | 86.4% |
| HWESASLLR and lysine | 94.8% | 89.7% | 87.7% |
| dihomolinoleate (20:2n6) and HWESASLLR | 95.8% | 89.1% | 87.2% |
| dihomolinolenate (20:3n3 or 3n6) and HWESASLLR | 95.8% | 90.6% | 85.7% |
| HWESASLLR and linoleate (18:2n6) | 95.4% | 88.9% | 87.6% |
| gamma-glutamylalanine and HWESASLLR | 95.5% | 90.6% | 85.7% |
| HWESASLLR and leucylalanine | 96.2% | 88.7% | 86.7% |
| glycylproline and HWESASLLR | 95.6% | 90.2% | 85.7% |
| HWESASLLR and oleate (18:1n9) | 96.0% | 89.3% | 86.2% |
| gamma-glutamyllysine and HWESASLLR | 95.1% | 89.9% | 86.4% |
| HWESASLLR and iminodiacetate (IDA) | 95.9% | 90.3% | 85.1% |
| HWESASLLR and succinate | 93.9% | 87.4% | 89.9% |
| HWESASLLR and leucylglutamate | 95.9% | 90.8% | 84.4% |
| HWESASLLR and isoleucylphenylalanine | 95.9% | 86.7% | 88.6% |
| HWESASLLR and linolenate (18:3n3 or 3n6) | 96.1% | 88.8% | 86.1% |
| glycolate (hydroxyacetate) and HWESASLLR | 95.7% | 90.0% | 85.2% |
| HWESASLLR and salicylate | 95.7% | 90.6% | 84.3% |
| adenine and HWESASLLR | 95.2% | 91.2% | 84.0% |
| HWESASLLR and isoleucine | 95.8% | 87.1% | 87.4% |
| HWESASLLR and methionine | 94.5% | 90.7% | 85.0% |
| 6-oxopiperidine-2-carboxylic acid and HWESASLLR | 95.3% | 88.4% | 86.3% |
| gamma-glutamylmethionine and HWESASLLR | 95.1% | 89.8% | 85.1% |
| histidine and HWESASLLR | 95.1% | 89.7% | 85.2% |
| HWESASLLR and pyrophosphate (PPi) | 95.5% | 89.6% | 84.8% |
| HWESASLLR and myo-inositol | 94.9% | 90.7% | 84.2% |
| 10-heptadecenoate (17:1n7) and HWESASLLR | 95.5% | 89.7% | 84.3% |
| HWESASLLR and suberate (octanedioate) | 95.5% | 89.9% | 84.1% |
| glycerol and HWESASLLR | 95.0% | 90.0% | 84.4% |
| 1-eicosapentaenoylglycerophosphoethanolamine and | 95.7% | 90.1% | 83.6% |
| HWESASLLR | | | |
| 3-dehydrocarnitine and HWESASLLR | 94.9% | 91.0% | 83.2% |
| HWESASLLR and sphingosine 1-phosphate | 95.7% | 91.1% | 82.2% |
| 5alpha-androstan-3,17-diol monosulfate (alpha,beta or beta,alpha) | 94.0% | 90.2% | 84.7% |
| and HWESASLLR | | | |
| docosadienoate (22:2n6) and HWESASLLR | 95.5% | 88.8% | 84.4% |
| N-oleoyltaurine and sarcosine (N-methylglycine) | 93.0% | 92.8% | 82.8% |
| 1-methylguanosine and HWESASLLR | 94.9% | 87.8% | 85.8% |
| 1-stearoylglycerol (18:0) and HWESASLLR | 95.1% | 91.4% | 81.9% |
| eicosenoate (20:1n9 or 1n11) and HWESASLLR | 95.8% | 88.8% | 83.8% |
| dihydroferulic acid and HWESASLLR | 95.4% | 91.2% | 81.1% |
| 3-methylglutaroylcarnitine (C6) and HWESASLLR | 95.3% | 90.9% | 81.6% |
| HWESASLLR and trimethylamine N-oxide | 95.3% | 89.3% | 83.1% |
| alanine and HWESASLLR | 94.6% | 89.2% | 83.8% |
| HWESASLLR and urate | 95.5% | 90.7% | 81.3% |
| HWESASLLR and pentadecanoate (15:0) | 95.4% | 89.0% | 83.1% |
| 10-nonadecenoate (19:1n9) and HWESASLLR | 95.6% | 89.4% | 82.4% |
| 5alpha-androstan-3alpha,17beta-diol monosulfate 1 and | 95.2% | 88.3% | 83.9% |
| HWESASLLR | | | |
| fumarate and HWESASLLR | 93.9% | 86.2% | 87.2% |
| docosapentaenoate (DPA; 22:5n3) and HWESASLLR | 94.9% | 89.8% | 82.6% |
| HWESASLLR and palmitoleate (16:1n7) | 95.5% | 90.1% | 81.6% |
| HWESASLLR and vaccenate (18:1n7) | 94.6% | 87.9% | 84.7% |
| HWESASLLR and leucine | 94.8% | 88.7% | 83.7% |
| HWESASLLR and methylpalmitate (15 or 2) | 94.9% | 88.9% | 83.3% |
| HWESASLLR and propionylcarnitine (C3) | 95.2% | 88.9% | 83.0% |
| 3-hydroxydecanoate and HWESASLLR | 95.1% | 88.4% | 83.6% |
| 3-[3-(sulfooxy)phenyl]propanoic acid and HWESASLLR | 94.1% | 92.7% | 80.2% |
| HWESASLLR and piperine | 94.9% | 88.3% | 83.7% |
| 3-carboxy-4-methyl-5-propyl-2-furanpropanoate (CMPF) and | 95.5% | 89.0% | 82.3% |
| HWESASLLR | | | |
| 2-hydroxypalmitate and HWESASLLR | 94.3% | 86.0% | 86.4% |
| HWESASLLR and ornithine | 95.0% | 89.9% | 81.8% |
| 3-hydroxybutyrate (BHBA) and HWESASLLR | 95.0% | 89.8% | 81.8% |
| HWESASLLR and N2,N5-diacetylornithine | 95.0% | 89.0% | 82.4% |
| HWESASLLR and myristate (14:0) | 95.0% | 88.7% | 82.7% |
| 4-androsten-3beta,17beta-diol monosulfate 2 and HWESASLLR | 94.8% | 90.1% | 81.4% |
| HWESASLLR and taurolithocholate 3-sulfate | 94.8% | 90.4% | 81.0% |
| HWESASLLR and laurylcarnitine (C12) | 94.5% | 89.8% | 81.9% |
| HWESASLLR and N-palmitoyltaurine | 95.1% | 89.1% | 82.0% |
| HWESASLLR and L-urobilin | 95.3% | 89.8% | 80.8% |
| ergothioneine and HWESASLLR | 94.5% | 87.3% | 84.0% |
| gamma-glutamylglutamate and HWESASLLR | 94.8% | 90.7% | 80.2% |
| HWESASLLR and laurate (12:0) | 94.8% | 90.4% | 80.3% |
| HWESASLLR and margarate (17:0) | 95.5% | 87.0% | 82.9% |
| HWESASLLR and palmitoylcarnitine (C16) | 95.4% | 89.6% | 80.3% |
| HWESASLLR and oleoylcarnitine (C18) | 94.5% | 89.0% | 81.7% |
| 5alpha-androstan-3beta,17beta-diol monosulfate 2 and | 95.0% | 88.4% | 81.7% |
| HWESASLLR | | | |
| HWESASLLR and valylglutamine | 94.9% | 89.6% | 80.7% |
| 5-dodecenoate (12:1n7) and HWESASLLR | 94.4% | 90.0% | 80.6% |
| HWESASLLR and pyruvate | 94.5% | 90.2% | 80.1% |
| caprate (10:0) and HWESASLLR | 94.8% | 89.7% | 80.2% |
| 2-hydroxybutyrate (AHB) and HWESASLLR | 94.4% | 89.1% | 81.1% |
| caprylate (8:0) and HWESASLLR | 94.6% | 88.2% | 81.7% |
| 17-methylstearate and HWESASLLR | 94.8% | 87.9% | 81.8% |
| HWESASLLR and phenylacetate | 94.4% | 88.6% | 81.4% |
| adrenate (22:4n6) and HWESASLLR | 95.1% | 88.7% | 80.7% |
| HWESASLLR and nonadecanoate (19:0) | 95.2% | 87.8% | 81.3% |
| HWESASLLR and tetradecanedioate (C14) | 95.0% | 88.1% | 81.2% |
| HWESASLLR and N-acetylcarnosine | 94.8% | 85.4% | 84.0% |
| HWESASLLR and methyl-beta-glucopyranoside | 94.5% | 89.0% | 80.6% |
| citmte and HWESASLLR | 95.1% | 85.1% | 83.2% |
| HWESASLLR and N-acetylglycine | 94.4% | 88.1% | 80.6% |
| hexadecanedioate (C16) and HWESASLLR | 94.7% | 87.3% | 80.4% |
| HWESASLLR and propionylglycine (C3) | 93.7% | 86.8% | 81.8% |
| gamma-glutamylmethionine and sarcosine (N-methylglycine) | 87.7% | 90.6% | 81.0% |
| gamma-glutamyllysine and iminodiacetate (IDA) | 89.9% | 86.3% | 81.7% |
| margarate (17:0) and sarcosine (N-methylglycine) | 89.7% | 87.0% | 80.9% |
| linoleate (18:2n6) and sarcosine (N-methylglycine) | 87.7% | 85.7% | 84.1% |
| gamma-glutamylmethionine and iminodiacetate (IDA) | 87.9% | 86.4% | 81.6% |
| sarcosine (N-methylglycine) and suberate (octanedioate) | 86.5% | 83.9% | 84.3% |
| hypoxanthine and tetradecanedioate (C14) | 89.0% | 84.8% | 80.9% |
| 10-nonadecenoate (19:1n9) and sarcosine (N-methylglycine) | 88.7% | 85.0% | 80.9% |
| docosadienoate (22:2n6) and sarcosine (N-methylglycine) | 88.8% | 83.4% | 82.2% |
| 13-methylmyristic acid and sarcosine (N-methylglycine) | 88.1% | 82.7% | 83.6% |
| eicosenoate (20:1n9 or 1n11) and sarcosine (N-methylglycine) | 88.0% | 84.8% | 81.4% |
| methionine and sarcosine (N-methylglycine) | 86.9% | 86.1% | 80.9% |
| myristoleate (14:1n5) and sarcosine (N-methylglycine) | 87.9% | 82.1% | 83.6% |
| sarcosine (N-methylglycine) and tetradecanedioate (C14) | 87.9% | 84.7% | 80.9% |
| 17-methylstearate and sarcosine (N-methylglycine) | 88.4% | 80.8% | 83.9% |
| oleate (18:1n9) and sarcosine (N-methylglycine) | 88.4% | 84.4% | 80.2% |
| dihomolinoleate (20:2n6) and sarcosine (N-methylglycine) | 87.3% | 83.1% | 81.8% |
| methylpalmitate (15 or 2) and sarcosine (N-methylglycine) | 89.3% | 81.9% | 80.6% |
| iminodiacetate (IDA) and isoleucine | 86.8% | 83.1% | 81.6% |
| gamma-glutamylalanine and sarcosine (N-methylglycine) | 87.3% | 82.3% | 81.0% |
| gamma-glutamylglutamate and sarcosine (N-methylglycine) | 85.5% | 82.6% | 82.2% |
| palmitoylcarnitine (C16) and sarcosine (N-methylglycine) | 87.9% | 80.8% | 81.0% |
| palmitoleate (16:1n7) and sarcosine (N-methylglycine) | 88.0% | 80.9% | 80.4% |
| 10-heptadecenoate (17:1n7) and sarcosine (N-methylglycine) | 87.5% | 80.1% | 81.1% |
| isoleucine and sarcosine (N-methylglycine) | 86.1% | 80.3% | 81.4% |
| 3-[3-(sulfooxy)phenyl]propanoic acid and myo-inositol | 84.1% | 82.9% | 80.1% |
| phenylacetate and sarcosine (N-methylglycine) | 84.7% | 80.4% | 81.3% |
| adrenate (22:4n6) and sarcosine (N-methylglycine) | 85.4% | 80.3% | 80.2% |
| 6-oxopiperidine-2-carboxylic acid and sarcosine (N- | 84.6% | 80.1% | 81.1% |
| methylglycine) | | | |
| HWESASLLR and inosine | 96.1% | 91.6% | 86.9% |
| HWESASLLR and tryptophan | 94.9% | 88.7% | 86.3% |
| HWESASLLR and tyrosine | 95.4% | 89.4% | 84.1% |
| hippurate and HWESASLLR | 94.9% | 89.6% | 82.1% |
| HWESASLLR and isovalerate (C5) | 94.9% | 90.1% | 81.2% |
| 1-palmitoylglycerol (16:0) and HWESASLLR | 94.6% | 88.4% | 82.8% |
| dodecanedioate (C12) and HWESASLLR | 95.0% | 88.9% | 80.6% |
| glutaroylcarnitine (C5) and HWESASLLR and | 97.8% | 94.4% | 94.3% |
| phenylalanylalanine | | | |
| glutaroylcarnitine (C5) and HWESASLLR and isoleucylleucine | 97.7% | 93.9% | 94.8% |
| glutaroylcarnitine (C5) and HWESASLLR and myristoylcarnitine | 97.6% | 93.3% | 94.7% |
| glutaroylcarnitine (C5) and HWESASLLR and octadecanedioate | 97.2% | 94.0% | 93.7% |
| (C18) | | | |
| carnitine and glutaroylcarnitine (C5) and HWESASLLR | 96.9% | 94.2% | 92.7% |
| asparagylleucine and glutaroylcarnitine (C5) and HWESASLLR | 96.8% | 93.9% | 92.2% |
| 3-hydroxyoctanoate and glutaroylcarnitine (C5) and | 96.7% | 93.4% | 92.6% |
| HWESASLLR | | | |
| glutaroylcarnitine (C5) and HWESASLLR and taurine | 97.4% | 94.7% | 90.3% |
| 5-hydroxyindoleacetate and glutaroylcarnitine (C5) and | 96.9% | 94.6% | 89.9% |
| HWESASLLR | | | |
| 3-hydroxysebacate and glutaroylcarnitine (C5) and HWESASLLR | 97.0% | 93.0% | 90.9% |
| 3-hydroxysebacate and cysteine-glutathione disulfide and | 96.2% | 93.8% | 90.7% |
| HWESASLLR | | | |
| HWESASLLR and myristoylcarnitine and sarcosine (N- | 95.5% | 93.8% | 90.6% |
| methylglycine) | | | |
| cysteine-glutathione disulfide and HWESASLLR and | 95.4% | 95.0% | 88.9% |
| isoleucylleucine | | | |
| cysteine-glutathione disulfide and HWESASLLR and taurine | 95.5% | 94.8% | 88.9% |
| asparagylleucine and cysteine-glutathione disulfide and | 96.7% | 92.7% | 88.9% |
| HWESASLLR | | | |
| 3-methyl-2-oxobutyrate and HWESASLLR and sphinganine | 96.6% | 94.6% | 87.0% |
| cysteine-glutathione disulfide and HWESASLLR and | 95.8% | 94.7% | 87.1% |
| octadecanedioate (C18) | | | |
| HWESASLLR and phenylalanylalanine and sarcosine (N- | 95.8% | 93.9% | 87.9% |
| methylglycine) | | | |
| 3-methyl-2-oxobutyrate and HWESASLLR and threonylleucine | 96.4% | 97.3% | 83.6% |
| 3-hydroxyoctanoate and HWESASLLR and threonylleucine | 95.9% | 99.0% | 81.9% |
| 5-hydroxyindoleacetate and HWESASLLR and sarcosine (N- | 95.6% | 94.3% | 86.4% |
| methylglycine) | | | |
| carnitine and HWESASLLR and threonylleucine | 95.7% | 97.0% | 81.6% |
| 3-methyl-2-oxobutyrate and 4-methylcatechol sulfate and | 93.4% | 86.3% | 80.2% |
| HWESASLLR | | | |
| 13-methylmyristic acid and 4-methylcatechol sulfate and sarcosine | 86.5% | 80.4% | 81.9% |
| (N-methylglycine) | | | |
| glutaroylcarnitine (C5) and HWESASLLR and sebacate | 97.2% | 98.8% | 89.4% |
| (decanedioate) | | | |
| glutaroylcarnitine (C5) and HWESASLLR and undecanedioate | 97.2% | 94.8% | 90.8% |
| cysteine-glutathione disulfide and HWESASLLR and sebacate | 96.4% | 96.0% | 87.4% |
| (decanedioate) | | | |
| HWESASLLR and threonylleucine and undecanedioate | 96.3% | 99.4% | 82.9% |
| HWESASLLR and N-acetyltryptophan and tryptophan | 96.4% | 90.8% | 90.6% |
| HWESASLLR and N-acetyltryptophan and tyrosine | 95.9% | 90.4% | 87.9% |
| C-glycosyltryptophan and HWESASLLR and inosine | 96.0% | 91.6% | 86.4% |
| 3-hydroxyhippurate and HWESASLLR and inosine | 95.1% | 90.9% | 86.3% |
| HWESASLLR and inosine and N-acetyltyrosine | 95.3% | 90.4% | 86.0% |
| C-glycosyltryptophan and HWESASLLR and tryptophan | 95.5% | 90.3% | 85.4% |
| 4-hydroxyhippurate and HWESASLLR and inosine | 95.2% | 90.4% | 85.3% |
| HWESASLLR and N-acetyltyrosine and tryptophan | 95.3% | 89.2% | 85.8% |
| 4-hydroxyhippurate and HWESASLLR and sebacate | 94.2% | 92.6% | 82.3% |
| (decanedioate) | | | |
| 1-palmitoylglycerol (16:0) and 3-hydroxyhippurate and | 93.3% | 90.0% | 81.3% |
| HWESASLLR | | | |

### BIBLIOGRAPHY

1 Citron M (2004) Strategies for disease modification in Alzheimer's disease. Nat. Rev. Neurosci. 5, 677-85.
2 Suh Y-H & Checler F (2002) Amyloid precursor protein, presenilins, and alpha-synuclein: molecular pathogenesis and pharmacological applications in Alzheimer's disease. Pharmacol. Rev. 54, 469-525.
3 McKhann G, Drachman D, Folstein M, Katzman R, Price D & Stadlan E M (1984) Clinical diagnosis of Alzheimer's disease: report of the NINCDS-ADRDA Work Group under the auspices of Department of Health and Human Services Task Force on Alzheimer's Disease. Neurology 34, 939-44.
4 American Psychiatric Association (1997) Diagnostic and Statistical Manual of Mental Disorders, 4th ed. (American Psychiatric Association, ed.) U.S. National Library of Medicine, Washington, D.C.
5 Petersen R C, Smith G E, Waring S C, Ivnik R J, Tangalos E G & Kokmen E (1999) Mild cognitive impairment: clinical characterization and outcome. Arch. Neurol. 56, 303-8.
6 Albert M S, DeKosky S T, Dickson D, Dubois B, Feldman H H, Fox N C, Gamst A, Holtzman D M, Jagust W J, Petersen R C, Snyder P J, Carrillo M C, Thies B & Phelps C H (2011) The diagnosis of mild cognitive impairment due to Alzheimer's disease: recommendations from the National Institute on Aging-Alzheimer's Association workgroups on diagnostic guidelines for Alzheimer's disease. Alzheimers. Dement. 7, 270-9.
7 Thambisetty M & Lovestone S (2010) Blood-based biomarkers of Alzheimer's disease: challenging but feasible. Biomark. Med. 4, 65-79.
8 Gustaw-Rothenberg K, Lerner A, Bonda D J, Lee H, Zhu X, Perry G & Smith M A (2010) Biomarkers in Alzheimer's disease: past, present and future. Biomark. Med. 4, 15-26.
9 Rupsingh R, Borrie M, Smith M, Wells J L & Bartha R (2011) Reduced hippocampal glutamate in Alzheimer disease. Neurobiol. Aging 32, 802-10.
10 Skoumalová A, Ivica J, Santorová P, Topinková E & Wilhelm J (2011) The lipid peroxidation products as possible markers of Alzheimer's disease in blood. Exp. Gerontol. 46, 38-42.
11 Gabelle A, Richard F, Gutierrez L-A, Schraen S, Delva F, Rouaud O, Buée L, Dartigues J-F, Touchon J, Lambert J-C & Ben C (2013) Plasma amyloid-β levels and prognosis in incident dementia cases of the 3-City Study. J. Alzheimers. Dis. 33, 381-91.
12 Pesini P, Pérez-Grijalba V, Monleón I, Boada M, Tárraga L, Martinez-Lage P, San-José I & Sarasa M (2012) Reliable Measurements of the β-Amyloid Pool in Blood Could Help in the Early Diagnosis of AD. Int. J. Alzheimers. Dis. 2012, 604141.
13 Toledo J B, Shaw L M & Trojanowski J Q (2013) Plasma amyloid beta measurements—a desired but elusive Alzheimer's disease biomarker. Alzheimers. Res. Ther. 5, 8.
14 Doecke J D, Laws S M, Faux N G, Wilson W, Burnham S C, Lam C-P, Mondal A, Bedo J, Bush A I, Brown B, De Ruyck K, Ellis K A, Fowler C, Gupta V B, Head R, Macaulay S L, Pertile K, Rowe C C, Rembach A, Rodrigues M, Rumble R, Szoeke C, Taddei K, Taddei T, Trounson B, Ames D, Masters C L & Martins R N (2012) Blood-based protein biomarkers for diagnosis of Alzheimer disease. Arch. Neurol. 69, 1318-25.
15 Aftab M F & Waraich R S (2012) A REVIEW OF BIOCHEMICAL MARKERS FOR EARLY DIAGNOSIS OF ALZHEIMER'™S DISEASE. Am. J. Neurosci. 3, 54-62.
16 Rosen C, Hansson O, Blennow K & Zetterberg H (2013) Fluid biomarkers in Alzheimer's disease—current concepts. Mol. Neurodegener. 8, 20.
17 Gabelle A, Touchon J & Lehmann S (2013) Les biomarqueurs du LCR et du plasma: utilisation diagnostique et pronostique dans la maladie d'Alzheimer et les syndromes apparentés. Prat. Neurol.—FMC 4, 65-72.
18 Laxman Kole P, Venkatesh G, Kotecha J & Sheshala R (2011) Recent advances in sample preparation techniques for effective bioanalytical methods. Biomed. Chromatogr. 25, 199-217.
19 Sabbagh M N, Fleisher A, Chen K, Rogers J, Berk C, Reiman E, Pontecorvo M, Mintun M, Skovronsky D, Jacobson S A, Sue L I, Liebsack C, Charney A S, Cole L, Belden C & Beach T G (2011) Positron emission tomography and neuropathologic estimates of fibrillar amyloid-β in a patient with Down syndrome and Alzheimer disease. Arch. Neurol. 68, 1461-6.
20 Babu S V S, Shareef M M, Shetty A P K & Shetty K T (2002) HPLC method for amino acids profile in biological fluids and inborn metabolic disorders of aminoacidopathies. Indian J. Clin. Biochem. 17, 7-26.
21 Lima E. S. & Abdalla D. S. P. High-performance liquid chromatography of fatty acids in biological samples. Anal. Chim. Acta 465, 11.
22 Chen S.-H. & Chuang Y.-J. Analysis of fatty acids by column liquid chromatography. Anal. Chim. Acta 465, 11.
23 Bondia-Pons I, Castellote A I & Lopez-Sabater M C (2004) Comparison of conventional and fast gas chromatography in human plasma fatty acid determination. J. Chromatogr. B. Analyt. Technol. Biomed. Life Sci. 809, 339-44.
24 Stoltenburg R, Reinemann C & Strehlitz B (2007) SELEX—a (r)evolutionary method to generate high-affinity nucleic acid ligands. Biomol. Eng. 24, 381-403.
25 Radom F, Jurek P M, Mazurek M P, Otlewski J & Jelen F (2013) Aptamers: Molecules of great potential. Biotechnol. Adv. 31, 1260-1274.
26 Kuhn A, Luthi-Carter R & Delorenzi M (2008) Cross-species and cross-platform gene expression studies with the Bioconductor-compliant R package “annotationTools”. BMC Bioinformatics 9, 26.
27 Chong I-G & Jun C-H (2005) Performance of some variable selection methods when multicollinearity is present. Chemom. Intell. Lab. Syst. 78, 103-112.
28 Wold H (1966) Estimation of principal components and related models by iterative least squares. J. Multivar. Anal.
29 Wold S, Johansson E & Cocchi M (1993) 3D-QSAR in Drug Design: Theory, Methods and Applications. (E. Science, ed.) Ledien.
30 Azizan K A, Baharum S N, Ressom H W & Noor N M (2012) GC-MS Analysis and PLS-DA Validation of the Trimethyl Silyl-Derivatization Techniques. Am. J. Appl. Sci. 9, 1124-1136.
31 McLachlan G J (2004) Discriminant Analysis and Statistical Pattern Recognition (J. Wiley, ed.) Wiley Interscience.

1-15. (canceled)

16. An in vitro method for diagnosing a neurological disease selected from Alzheimer's disease (AD), senile dementia of AD type (SDAT), prodromal AD, mild cognitive impairment (MCI), age associated memory impairment (AAMI), vascular dementia or frontotemporal dementia (FTD) in a subject, the method comprising determining, in a sample of blood, serum and/or plasma from said subject, the presence, quantity, frequency or form of one or more biomarker(s) selected from iminodiacetate (IDA); sarcosine (N methylglycine); HWESASLLR; 3-[3-(sulfooxy)phenyl]propanoic acid; leucylglycine; tetradecanedioate (C14); 3-hydroxybutyrate (BHBA); hexadecanedioate (C16); 3-dehydrocarnitine; caprate (10:0); threonylleucine; leucylglutamate; leucylalanine; N-oleoyltaurine; 2-hydroxybutyrate (AHB); a mix of 13-HODE and 9-HODE; sphinganine; hypoxanthine; glycolate (hydroxyacetate); taurocholenate sulfate; phenylacetate; myristate (14:0); margarate (17:0); valylglutamine; stearate (18:0); N-palmitoyltaurine; hydroxybutyrylcarnitine; glycerol; gamma-glutamyl alanine; piperine; laurate (12:0); 10-nonadecenoate (19:1n9); dihomolinoleate (20:2n6); eicosenoate (20:1n9 or 1n11); lysine; 3-hydroxydecanoate; palmitate (16:0); 3-hydroxyhippurate; 5-dodecenoate (12:1n7); acetylcarnitine (C2); 5alpha-androstan-3beta,17beta-diol monosulfate 2; methyl-beta-glucopyranoside; 1-eicosapentaenoylglycerophosphocholine (20:5n3); docosadienoate (22:2n6); gamma-glutamylmethionine; methylpalmitate (15 or 2); pentadecanoate (15:0); docosatrienoate (22:3n3); myristoylcarnitine; linoleate (18:2n6); 1-stearoylglycerol (18:0); myristoleate (14:1n5); oleate (18:1n9); 13-methylmyristic acid; nonadecanoate (19:0); 10-heptadecenoate (17:1n7); 5alpha-androstan-3,17-diol monosulfate (alpha,beta or beta,alpha); 17-methylstearate; vaccenate (18:1n7); palmitoleate (16:1n7); adrenate (22:4n6); linolenate (18:3n3 or 3n6); docosapentaenoate (DPA; 22:5n3); dihomolinolenate (20:3n3 or 3n6); adenine; 3-hydroxysebacate; N-acetyltyrosine; octadecanedioate (C18); isoleucylleucine; ergothioneine; N-acetylglycine; caprylate (8:0); citrate; N-acetyltryptophan; palmitoyl ethanolamide; histidine; asparagylleucine; 4-methylcatechol sulfate; suberate (octanedioate); methionine; cysteine-glutathione disulfide; 6-oxopiperidine-2-carboxylic acid; glutaroylcarnitine (C5); taurolithocholate 3-sulfate; ornithine; palmitoylcarnitine (C16); 5alpha-androstan-3alpha,17beta-diol monosulfate 1; pyruvate; urate; 1-methylguanosine; C-glycosyltryptophan; 1-eicosapentaenoylglycerophosphoethanolamine; 3-hydroxyoctanoate; oleoylcarnitine (C18); sphingosine 1-phosphate; phenylalanylalanine; alanine; 3-methylglutaroylcarnitine (C6); N-acetylcarnosine; isoleucine; dihydroferulic acid; homovanillate sulfate; uridine; 4-hydroxyhippurate; leucine; glycylproline; trimethylamine N-oxide; laurylcarnitine (C12); propionylglycine (C3); propionylcarnitine (C3); fumarate; L-urobilin; glycerate; gamma-glutamyllysine; myo-inositol; pregnen-diol disulfate; 5-hydroxyindoleacetate; 2-hydroxypalmitate; 3-methyl-2-oxobutyrate; N2,N5-diacetylornithine; 4-androsten-3beta,17beta-diol monosulfate 2; taurine; valylvaline; gamma-glutamylglutamate; 3-carboxy-4-methyl-5-propyl-2-furanpropanoate (CMPF); carnitine; salicylate; succinate; isoleucylphenylalanine; riboflavin (Vitamin B2); and pyrophosphate (PPi), wherein an alteration in the presence, quantity, frequency or form of said one or more biomarker(s) as compared to a control is indicative of the presence, risk, subtype, progression or severity of said disease.

17. The in vitro method of claim 16, wherein said one or more biomarker(s) is selected from iminodiacetate (IDA); sarcosine (N-methylglycine); HWESASLLR; 3-[3-(sulfooxy)phenyl]propanoic acid; leucylglycine; tetradecanedioate (C14); 3-hydroxybutyrate (BHBA); hexadecanedioate (C16); 3-dehydrocarnitine; caprate (10:0); threonylleucine; leucylglutamate; leucylalanine; N-oleoyltaurine; 2-hydroxybutyrate (AHB); a mix of 13-HODE and 9-HODE; sphinganine; hypoxanthine; glycolate (hydroxyacetate); taurocholenate sulfate; phenylacetate; myristate (14:0); margarate (17:0); valylglutamine; stearate (18:0); N-palmitoyltaurine; hydroxybutyrylcamitine; glycerol; gamma-glutamylalanine; piperine; laurate (12:0); 10-nonadecenoate (19:1n9); dihomolinoleate (20:2n6); eicosenoate (20:1n9 or 1 n11); lysine; 3-hydroxydecanoate; palmitate (16:0); 3-hydroxyhippurate; 5-dodecenoate (12:1n7); acetylcarnitine (C2); 5alpha-androstan-3beta,17beta-diol monosulfate 2; methyl-beta-glucopyranoside; 1-eicosapentaenoylglycerophosphocholine (20:5n3); docosadienoate (22:2n6); gamma-glutamylmethionine; methylpalmitate (15 or 2); pentadecanoate (15:0); docosatrienoate (22:3n3); myristoylcarnitine; linoleate (18:2n6); 1-stearoylglycerol (18:0); myristoleate (14:1n5); oleate (18:1n9); 13-methylmyristic acid; nonadecanoate (19:0); 10-heptadecenoate (17:1n7); 5alpha-androstan-3,17-diol monosulfate (alpha,beta or beta,alpha); 17-methylstearate; vaccenate (18:1n7); palmitoleate (16:1n7); adrenate (22:4n6); linolenate (18:3n3 or 3n6); docosapentaenoate (DPA; 22:5n3); and dihomolinolenate (20:3n3 or 3n6).

18. The in vitro method of claim 16, the method comprising determining the presence, quantity, frequency or form, in a sample of blood, serum and/or plasma from said subject, of (i) one or more biomarker(s) selected from HWESASLLR and sarcosine (N-methylglycine), and (ii) one or more distinct biomarker(s) selected from iminodiacetate (IDA); sarcosine (N-methylglycine); HWESASLLR; 3-[3-(sulfooxy)phenyl]propanoic acid; leucylglycine; tetradecanedioate (C14); 3-hydroxybutyrate (BHBA); hexadecanedioate (C16); 3-dehydrocarnitine; caprate (10:0); threonylleucine; leucylglutamate; leucylalanine; N-oleoyltaurine; 2-hydroxybutyrate (AHB); a mix of 13-HODE and 9-NODE; sphinganine; hypoxanthine; glycolate (hydroxyacetate); taurocholenate sulfate; phenylacetate; myristate (14:0); margarate (17:0); valylglutamine; stearate (18:0); N-palmitoyltaurine; hydroxybutyrylcarnitine; glycerol; gamma-glutamylalanine; piperine; laurate (12:0); 10-nonadecenoate (19:1n9); dihomolinoleate (20:2n6); eicosenoate (20:1n9 or 1n11); lysine; 3-hydroxydecanoate; palmitate (16:0); 3-hydroxyhippurate; 5-dodecenoate (12:1n7); acetylcarnitine (C2); 5alpha-androstan-3beta,17beta-diol monosulfate 2; methyl-beta-glucopyranoside; 1-eicosapentaenoylglycerophosphocholine (20:5n3); docosadienoate (22:2n6); gamma-glutamylmethionine; methylpalmitate (15 or 2); pentadecanoate (15:0); docosatrienoate (22:3n3); myristoylcarnitine; linoleate (18:2n6); 1-stearoylglycerol (18:0); myristoleate (14:1n5); oleate (18:1n9); 13-methylmyristic acid; nonadecanoate (19:0); 10-heptadecenoate (17:1n7); 5alpha-androstan-3,17-diol monosulfate (alpha,beta or beta,alpha); 17-methylstearate; vaccenate (18:1n7); palmitoleate (16:1n7); adrenate (22:4n6); linolenate (18:3n3 or 3n6); docosapentaenoate (DPA; 22:5n3); and dihomolinolenate (20:3n3 or 3n6), wherein an alteration of said presence, quantity, frequency or form is indicative of the presence, risk, subtype, progression or severity of said disease.

19. The in vitro method of claim 16, wherein said one or more biomarkers comprise a set of at least two biomarkers selected from iminodiacetate (IDA), sarcosine (N-methylglycine), HWESASLLR, 3-[3-(sulfooxy)phenyl]propanoic acid, leucylglycine, tetradecanedioate (C14), 3-hydroxybutyrate (BHBA), hexadecanedioate (C16), 3-dehydrocarnitine, caprate (10:0), threonylleucine and leucylglutamate.

20. The in vitro method of claim 16, wherein said biomarker(s) are selected from iminodiacetate (IDA), sarcosine (N-methylglycine), HWESASLLR, and 3-[3-(sulfooxy)phenyl]propanoic acid.

21. The in vitro method of claim 16, wherein at least one of said one or more biomarkers is sarcosine (N-methylglycine).

22. The in vitro method of claim 16, wherein at least one of said one or more biomarkers is HWESASLLR.

23. The in vitro method of claim 16, wherein at least one of said one or more biomarkers is iminodiacetate (IDA).

24. The in vitro method of claim 16, wherein at least one of said one or more biomarkers is 3-[3-(sulfooxy)phenyl]propanoic acid.

25. The in vitro method of claim 16, further comprising the simultaneous or sequential determination of an alteration in the quantity, frequency or form of at least one additional biomarker or diagnostic test.

26. The method of claim 25, wherein the at least one additional diagnostic test or biomarker is selected from nucleic acids, proteins, metabolites, neurophysiological, genetic, brain imaging, clinical and cognitive tests or markers.

27. The method of claim 26, wherein said metabolites are selected from PFAM(20:1), PFAM(22:1), PFAM(22:2), hippurate, tyrosine, tryptophan, undecanedioate, isovalerate (C5), 1-palmitoylglycerol (16:0), dodecanedioate (C12), sebacate (decanedioate) and inosine.

28. The in vitro method of claim 16, comprising determining simultaneously or sequentially the presence of an alteration in the quantity, frequency or form of a set of biomarkers selected from:
iminodiacetate (IDA) and PFAM (22:1),
iminodiacetate (IDA) and PFAM (20:1),
iminodiacetate (IDA) and PFAM (22:2),
glutaroylcarnitine (C5) and HWESASLLR,
glycerate and HWESASLLR,
HWESASLLR and threonylleucine,
cysteine-glutathione disulfide and HWESASLLR,
HWESASLLR and hypoxanthine,
HWESASLLR and valylvaline,
HWESASLLR and palmitate (16:0),
HWESASLLR and sphinganine,
HWESASLLR and sarcosine (N-methylglycine),
homovanillate sulfate and HWESASLLR,
HWESASLLR and leucylglycine,
docosatrienoate (22:3n3) and HWESASLLR,
a mix of 13-HODE and 9-HODE and HWESASLLR,
HWESASLLR and palmitoyl ethanolamide,
acetylcarnitine (C2) and HWESASLLR,
HWESASLLR and taurocholenate sulfate,
HWESASLLR and riboflavin (Vitamin B2),
HWESASLLR and uridine,
HWESASLLR and pregnen-diol disulfate,
1-eicosapentaenoylglycerophosphocholine (20:5n3) and HWESASLLR,
HWESASLLR and stearate (18:0),
HWESASLLR and hydroxybutyrylcarnitine,
HWESASLLR and lysine,
dihomolinoleate (20:2n6) and HWESASLLR,
dihomolinolenate (20:3n3 or 3n6) and HWESASLLR,
HWESASLLR and linoleate (18:2n6),
gamma-glutamylalanine and HWESASLLR,
HWESASLLR and leucylalanine,
glycylproline and HWESASLLR,
HWESASLLR and oleate (18:1n9),
gamma-glutamyllysine and HWESASLLR,
HWESASLLR and iminodiacetate (IDA),
HWESASLLR and succinate,
HWESASLLR and leucylglutamate,
HWESASLLR and isoleucylphenylalanine,
HWESASLLR and linolenate (18:3n3 or 3n6),
glycolate (hydroxyacetate) and HWESASLLR,
HWESASLLR and salicylate,
adenine and HWESASLLR,
HWESASLLR and isoleucine,
HWESASLLR and methionine,
6-oxopiperidine-2-carboxylic acid and HWESASLLR,
gamma-glutamylmethionine and HWESASLLR,
histidine and HWESASLLR,
HWESASLLR and pyrophosphate (PPi),
HWESASLLR and myo-inositol,
10-heptadecenoate (17:1n7) and HWESASLLR,
HWESASLLR and suberate (octanedioate),
glycerol and HWESASLLR,
1-eicosapentaenoylglycerophosphoethanolamine and HWESASLLR,
3-dehydrocarnitine and HWESASLLR,
HWESASLLR and sphingosine 1-phosphate,
5alpha-androstan-3,17-diol monosulfate (alpha,beta or beta,alpha) and HWESASLLR,
docosadienoate (22:2n6) and HWESASLLR,
N-oleoyltaurine and sarcosine (N-methylglycine),
1-methylguanosine and HWESASLLR,
1-stearoylglycerol (18:0) and HWESASLLR,
eicosenoate (20:1n9 or 1n11) and HWESASLLR,
dihydroferulic acid and HWESASLLR,
3-methylglutaroylcarnitine (C6) and HWESASLLR,
HWESASLLR and trimethylamine N-oxide,
alanine and HWESASLLR,
HWESASLLR and urate,
HWESASLLR and pentadecanoate (15:0),
10-nonadecenoate (19:1n9) and HWESASLLR,
5alpha-androstan-3alpha,17beta-diol monosulfate 1 and HWESASLLR,
fumarate and HWESASLLR,
docosapentaenoate (DPA; 22:5n3) and HWESASLLR,
HWESASLLR and palmitoleate (16:1n7),
HWESASLLR and vaccenate (18:1n7),
HWESASLLR and leucine,
HWESASLLR and methylpalmitate (15 or 2),
HWESASLLR and propionylcarnitine (C3),
3-hydroxydecanoate and HWESASLLR,
3-[3-(sulfooxy)phenyl]propanoic acid and HWESASLLR,
HWESASLLR and piperine,
3-carboxy-4-methyl-5-propyl-2-furanpropanoate (CMPF) and HWESASLLR,
2-hydroxypalmitate and HWESASLLR,
HWESASLLR and ornithine,
3-hydroxybutyrate (BHBA) and HWESASLLR,
HWESASLLR and N2,N5-diacetylornithine,
HWESASLLR and myristate (14:0),
4-androsten-3beta,17beta-diol monosulfate 2 and HWESASLLR,
HWESASLLR and taurolithocholate 3-sulfate,
HWESASLLR and laurylcarnitine (C12),
HWESASLLR and N-palmitoyltaurine,
HWESASLLR and L-urobilin,
ergothioneine and HWESASLLR,
gamma-glutamylglutamate and HWESASLLR,
HWESASLLR and laurate (12:0),
HWESASLLR and margarate (17:0),
HWESASLLR and palmitoylcarnitine (C16),
HWESASLLR and oleoylcarnitine (C18),
5alpha-androstan-3beta,17beta-diol monosulfate 2 and HWESASLLR,
HWESASLLR and valylglutamine,
5-dodecenoate (12:1n7) and HWESASLLR,
HWESASLLR and pyruvate,
caprate (10:0) and HWESASLLR,
2-hydroxybutyrate (AHB) and HWESASLLR,
caprylate (8:0) and HWESASLLR,
17-methylstearate and HWESASLLR,
HWESASLLR and phenylacetate,
adrenate (22:4n6) and HWESASLLR,
HWESASLLR and nonadecanoate (19:0),
HWESASLLR and tetradecanedioate (C14),
HWESASLLR and N-acetylcarnosine,
HWESASLLR and methyl-beta-glucopyranoside,
citrate and HWESASLLR,
HWESASLLR and N-acetylglycine,
hexadecanedioate (C16) and HWESASLLR,
HWESASLLR and propionylglycine (C3),
gamma-glutamylmethionine and sarcosine (N-methylglycine),
gamma-glutamyllysine and iminodiacetate (IDA),
margarate (17:0) and sarcosine (N-methylglycine),
linoleate (18:2n6) and sarcosine (N-methylglycine),
gamma-glutamylmethionine and iminodiacetate (IDA),
sarcosine (N-methylglycine) and suberate (octanedioate),
hypoxanthine and tetradecanedioate (C14),
10-nonadecenoate (19:1n9) and sarcosine (N-methylglycine),
docosadienoate (22:2n6) and sarcosine (N-methylglycine),
13-methylmyristic acid and sarcosine (N-methylglycine),
eicosenoate (20:1n9 or 1n11) and sarcosine (N-methylglycine),
methionine and sarcosine (N-methylglycine),
myristoleate (14:1n5) and sarcosine (N-methylglycine),
sarcosine (N-methylglycine) and tetradecanedioate (C14),
17-methylstearate and sarcosine (N-methylglycine),
oleate (18:1n9) and sarcosine (N-methylglycine),
dihomolinoleate (20:2n6) and sarcosine (N-methylglycine),
methylpalmitate (15 or 2) and sarcosine (N-methylglycine),
iminodiacetate (IDA) and isoleucine,
gamma-glutamylalanine and sarcosine (N-methylglycine),
gamma-glutamylglutamate and sarcosine (N-methylglycine),
palmitoylcarnitine (C16) and sarcosine (N-methylglycine),
palmitoleate (16:1n7) and sarcosine (N-methylglycine),
10-heptadecenoate (17:1n7) and sarcosine (N-methylglycine),
isoleucine and sarcosine (N-methylglycine),
3-[3-(sulfooxy)phenyl]propanoic acid and myo-inositol,
phenylacetate and sarcosine (N-methylglycine),
adrenate (22:4n6) and sarcosine (N-methylglycine),
6-oxopiperidine-2-carboxylic acid and sarcosine (N-methylglycine),
glutaroylcarnitine (C5) and HWESASLLR and phenylalanylalanine,
glutaroylcarnitine (C5) and HWESASLLR and isoleucylleucine,
glutaroylcarnitine (C5) and HWESASLLR and myristoylcarnitine,
glutaroylcarnitine (C5) and HWESASLLR and octadecanedioate (C18),
carnitine and glutaroylcarnitine (C5) and HWESASLLR,
asparagylleucine and glutaroylcarnitine (C5) and HWESASLLR,
3-hydroxyoctanoate and glutaroylcarnitine (C5) and HWESASLLR,
glutaroylcarnitine (C5) and HWESASLLR and taurine,
5-hydroxyindoleacetate and glutaroylcarnitine (C5) and HWESASLLR,
3-hydroxysebacate and glutaroylcarnitine (C5) and HWESASLLR,
3-hydroxysebacate and cysteine-glutathione disulfide and HWESASLLR,
HWESASLLR and myristoylcarnitine and sarcosine (N-methylglycine),
cysteine-glutathione disulfide and HWESASLLR and isoleucylleucine,
cysteine-glutathione disulfide and HWESASLLR and taurine,
asparagylleucine and cysteine-glutathione disulfide and HWESASLLR,
3-methyl-2-oxobutyrate and HWESASLLR and sphinganine,
cysteine-glutathione disulfide and HWESASLLR and octadecanedioate (C18),
HWESASLLR and phenylalanylalanine and sarcosine (N-methylglycine),
3-methyl-2-oxobutyrate and HWESASLLR and threonylleucine,
3-hydroxyoctanoate and HWESASLLR and threonylleucine,
5-hydroxyindoleacetate and HWESASLLR and sarcosine (N-methylglycine),
carnitine and HWESASLLR and threonylleucine,
3-methyl-2-oxobutyrate and 4-methylcatechol sulfate and HWESASLLR,
13-methylmyristic acid and 4-methylcatechol sulfate and sarcosine (N-methylglycine),
HWESASLLR and PFAM (22:1),
HWESASLLR and PFAM (20:1),
HWESASLLR and PFAM (22:2),
sarcosine (N-methylglycine) and PFAM (22:1),
sarcosine (N-methylglycine) and PFAM (20:1),
sarcosine (N-methylglycine) and PFAM (22:2),
3-[3-(sulfooxy)phenyl]propanoic acid and PFAM (22:1),
3-[3-(sulfooxy)phenyl]propanoic acid and PFAM (20:1),
3-[3-(sulfooxy)phenyl]propanoic acid and PFAM (22:2),
HWESASLLR and inosine,
HWESASLLR and tryptophan,
HWESASLLR and tyrosine,
hippurate and HWESASLLR,
HWESASLLR and isovalerate (C5),
1-palmitoylglycerol (16:0) and HWESASLLR,
dodecanedioate (C12) and HWESASLLR,
glutaroylcarnitine (C5) and HWESASLLR and sebacate (decanedioate),
glutaroylcarnitine (C5) and HWESASLLR and undecanedioate,
cysteine-glutathione disulfide and HWESASLLR and sebacate (decanedioate),
HWESASLLR and threonylleucine and undecanedioate,
HWESASLLR and N-acetyltryptophan and tryptophan,
HWESASLLR and N-acetyltryptophan and tyrosine,
C-glycosyltryptophan and HWESASLLR and inosine,
3-hydroxyhippurate and HWESASLLR and inosine,
HWESASLLR and inosine and N-acetyltyrosine,
C-glycosyltryptophan and HWESASLLR and tryptophan,
4-hydroxyhippurate and HWESASLLR and inosine,
HWESASLLR and N-acetyltyrosine and tryptophan,
4-hydroxyhippurate and HWESASLLR and sebacate (decanedioate), or
1-palmitoylglycerol (16:0) and 3-hydroxyhippurate and HWESASLLR.

29. A method of treating a subject having or suspected to have a neurological disease selected from Alzheimer's disease (AD), senile dementia of AD type, prodromal AD, mild cognitive impairment, age associated memory impairment, vascular dementia or frontotemporal dementia, the method comprising (i) determining the presence, risk, subtype, progression or severity of said disease in a subject using a method of claim 16 and, (ii) administering to the subject in need thereof, a treatment against said disease.

30. An in vitro method for diagnosing a neurological disease selected from Alzheimer's disease (AD), senile dementia of AD type, prodromal AD, mild cognitive impairment, age associated memory impairment, vascular dementia or frontotemporal dementia, said method comprising the following steps:
collecting blood, serum or plasma sample from a subject suffering from, or suspected to suffer from, or at risk of suffering from said disease,
treating samples for their further analysis by LC/MS and/or GC/MS,
measuring by LC/MS and/or GC/MS an increase, as compared to a control value, of at least one biomarker selected from iminodiacetate (IDA), sarcosine (N-methylglycine), leucylglycine, tetradecanedioate (C14), 3-hydroxybutyrate (BHBA), hexadecanedioate (C16), 3-dehydrocarnitine, caprate (10:0), threonylleucine and leucylglutamate, and/or a decrease, as compared to a control value, of at least one biomarker selected from HWESASLLR and 3-[3-(sulfooxy)phenyl]propanoic acid, and
deducing from the preceding step the presence, risk, subtype, progression or severity of said disease.
